(19) United States
(12) Patent Application Publication
(10) Pub. No.: US 2011/0287146 A1
(43) Pub. Date: Nov. 24, 2011

(54) PACKAGING ASSEMBLY

(75) Inventors: Curtis H. Stowe, Gretna, NE (US); Kasey Ann Fuller, Council Bluffs, IA (US); Robert Donald Weick, Omaha, NE (US); Darrell Dragoo, Omaha, NE (US); Bart Hendryx, Omaha, NE (US); Jordan R. French, Evansville, IN (US); Sabrina Chan, Elgin, IL (US); Lori Cerwin, Gurnee, IL (US)

(73) Assignee: ConAgra Foods RDM, Inc., Omaha, NE (US)

(21) Appl. No.: 12/782,866

(22) Filed: May 19, 2010

(51) Int. Cl. B65D 85/00; G01N 33/02

(52) U.S. Cl. 426/88; 206/525; 426/124; 426/87

(57) ABSTRACT

A packaging nexus that drives a predetermined message to facilitate a consumer message is described. The nexus formed from the one or more components of the packaging assembly is configured to collectively signal and/or reinforce a predetermined message and at least partially facilitate a consumer impression.

### BACKGROUND

A consumer decision on whether to purchase a product can be partially influenced by the consumer's impression of the product at the point-of-sale. A consumer decision on whether to repurchase a product can be partially influenced by recollections of the impression of the product along with recollections of impressions during the use of the product after purchase.

### SUMMARY

This Summary is provided to introduce a selection of concepts in a simplified form that are further described below in the Detailed Description. This Summary is not intended to identify key and/or essential features of the claimed subject matter. Also, this Summary is not intended to limit the scope of the claimed subject matter in any manner.

Aspects of the disclosure pertain to a packaging assembly that includes a nexus between one or more elements of the packaging assembly. The nexus formed from the one or more components of the packaging assembly is configured to collectively signal and/or reinforce a predetermined message and at least partially facilitate a consumer impression.

### BRIEF DESCRIPTION OF THE DRAWINGS

FIG. 1 is an example perspective of the packaging assembly that illustrates the packaging assembly in a sealed state.

FIG. 2 is an example perspective of the packaging assembly of FIG. 1 that illustrates the packaging assembly prior to being sealed.

FIG. 3A is an example perspective of the container of the packaging assembly.

FIG. 3B is an example perspective of a second embodiment of a container of the packaging assembly.

FIG. 4 is an example top side of the container of the packaging assembly.

FIG. 5 is an example bottom side of the container of the packaging assembly.

FIG. 6 is an example dimensional diagram indicating the rim outer edge of the container.

FIG. 7 is an example dimensional diagram indicating the rim inner edge of the container.

FIG. 8 is an example first side of the container of the packaging assembly.

FIG. 9 is an example second side of the container of the packaging assembly.

FIG. 10 is an example third side of the container of the packaging assembly.

FIG. 11 is an example fourth side of the container of the packaging assembly.

FIG. 12 is an example cross-sectional view of the container taken along lines A-A in FIG. 4.

FIG. 13 is magnified view of area B in FIG. 12.

FIG. 14 is an example structural configuration of the sleeve of the packaging assembly

FIG. 15 is an example indicator field diagram associated with the sleeve of the packaging assembly.

FIG. 16 is an example sleeve front with example indicators populated in the indicator fields of FIG. 15.

FIG. 17 is an example sleeve back with example indicators populated in the indicator fields of FIG. 15.

FIG. 18 is an example sleeve first side with example indicators populated in the indicator fields of FIG. 15.

FIG. 19 is an example sleeve second side with example indicators populated in the indicator fields of FIG. 15.

FIG. 20 is an example sleeve third side with example indicators populated in the indicator fields of FIG. 15.

FIG. 21 is an example sleeve fourth side with example indicators populated in the indicator fields of FIG. 15.

### DETAILED DESCRIPTION

Aspects of the disclosure are described more fully hereinafter with reference to the accompanying drawings, which form a part hereof, and which show, by way of illustration, example features. The features can, however, be embodied in many different forms and should not be construed as limited to the combinations set forth herein; rather, these combinations are provided so that this disclosure will be thorough and complete, and will fully convey the scope. Among other things, the features of the disclosure can be facilitated by methods, devices, and/or embodied in articles of commerce. The following detailed description is, therefore, not to be taken in a limiting sense.

Dimensions are used throughout the description for exemplary purposes only. The description is not limited to any particular dimension set forth herein. For example, dimensions associated with the packaging assembly 100 can vary depending on desired size and shape of the packaging assembly 100. Moreover, the dimensions of any one of the components of packaging assembly 100 can be factored by a common multiplier to maintain the general shape of packaging assembly 100 while increasing or decreasing the respective size of packaging assembly 100.

Indicia is described and indicated throughout the specification and the figures. This description is not limited to any particular indicia type. Indicia can include structural indicia, textual indicia, image indicia and combinations thereof. Structural indicia can include a packaging configuration and/or shape. Other types of structural indicia can include one or more structural portions of container 200. Still other types of structural indicia can include one or more structural portions of sleeve 300. Yet other types of structural indicia can include a combination of structure from container 200 and sleeve 300. Textual indicia can include letters, words, sentences, symbols, numbers, dialog and combinations thereof. Image indicia can include photographs, pictures, drawings, paintings, holograms, icons and combinations thereof.

Consumer impressions can facilitate a purchase of any type of product. Yet, consumer impressions of a product can play a heightened role in relation to the purchase of transformative consumer products. A transformative consumer product is a product that includes multiple states from production to ultimate use. For example, a packaged food product can be considered a transformative consumer product. As other examples, refrigerated food products, prepared meals, box meals and the like can be transformative consumer products.

As an example associated with a food product at production, ingredients can be obtained in a prepackaged state. The ingredients can then be transformed to create a food product. The food product can then be transformed by packaging and shipping to a point-of-sale. After purchase, the food product is again transformed into a cooked food product ready for consumption. For example, the food product can be cooked in a microwave, steamed in a microwave, pressure cooked in a microwave, and combinations thereof. As other examples, the food product can be cooked via a stove, a grill or a conventional oven. In such a situation, consumer impressions play a heightened role in relation to the transformative consumer product because the preprocessed ingredients used to form the product can be perceived quite different in a prepackaged state as opposed to a packaged state. Also, the packaged state of the food product can be perceived quite different than the cooked state of the product. Accordingly, given that a user experience with the food product may not be readily ascertainable when the product is in the packaged state, an opportunity arises to frame or mold a perception of such an experience via a nexus of the packaging arrangement that drives a predetermined message.

As more fully set forth below, the disclosure herein pertains to a packaging nexus between one or more components of a packaging assembly. The nexus formed from the one or more components of the packaging assembly is configured to collectively signal and/or drive a predetermined message from the point-of-sale to the ultimate consumption of the consumer product.

Packaging Assembly

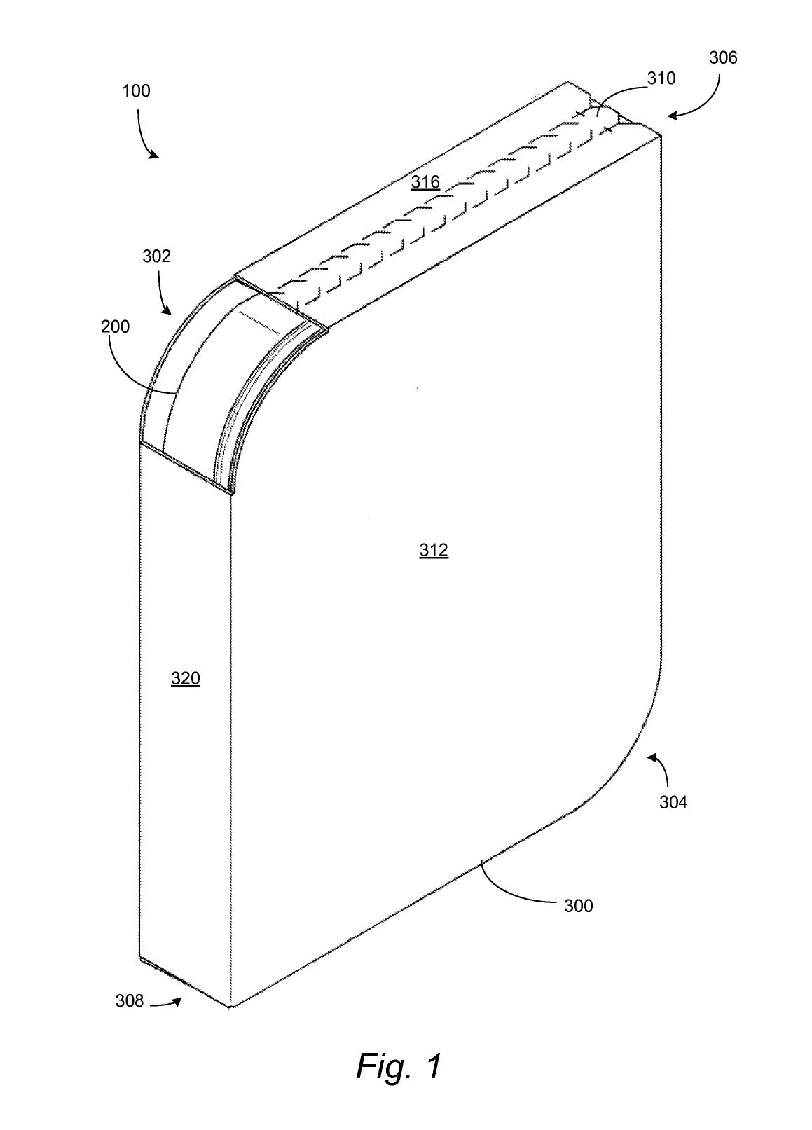

FIG. 1 is an example perspective of packaging assembly 100 that illustrates packaging assembly 100 in a sealed state. As more fully set forth below, packaging assembly 100 includes container 200 and sleeve 300. Container 200 can include a container suitable for cooking. For example, container 200 can include a microwave container, oven container, stove top container, and the like. Container 200 can be formed from a microwavable material such as a plastic. In other aspects, container 200 can be made of a paper composition, metal composition or other material suitable for cooking a food product. Sleeve 300 can include a substrate for facilitating an indicia and structural nexus for conveying a predetermined message. Sleeve 300 can include a substrate formed from a paper component, a laminated paper component, a plastic component and the like. Sleeve 300 can be arranged to at least partially enclose container 200. Sleeve 300 can include first corner opening 302 and second corner opening 304. First corner opening 302 and second corner opening 304 provide an opening so that a consumer can view container 200 through sleeve 300. Such corner openings 302 and 304 can further distinguish packaging arrangement 100 from other fully enclosed packages that may be located on a shelf at the point-of-sale. First corner opening 302 and second corner opening 304 are further arranged as set forth below to create a nexus between container 200 and sleeve 300 to drive a predetermined message.

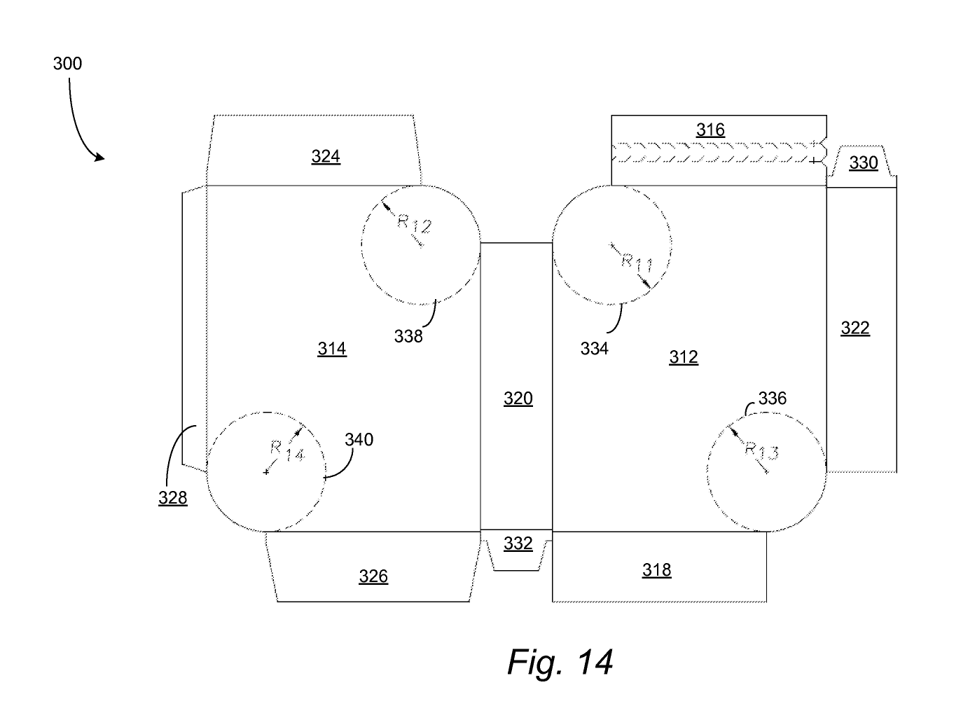

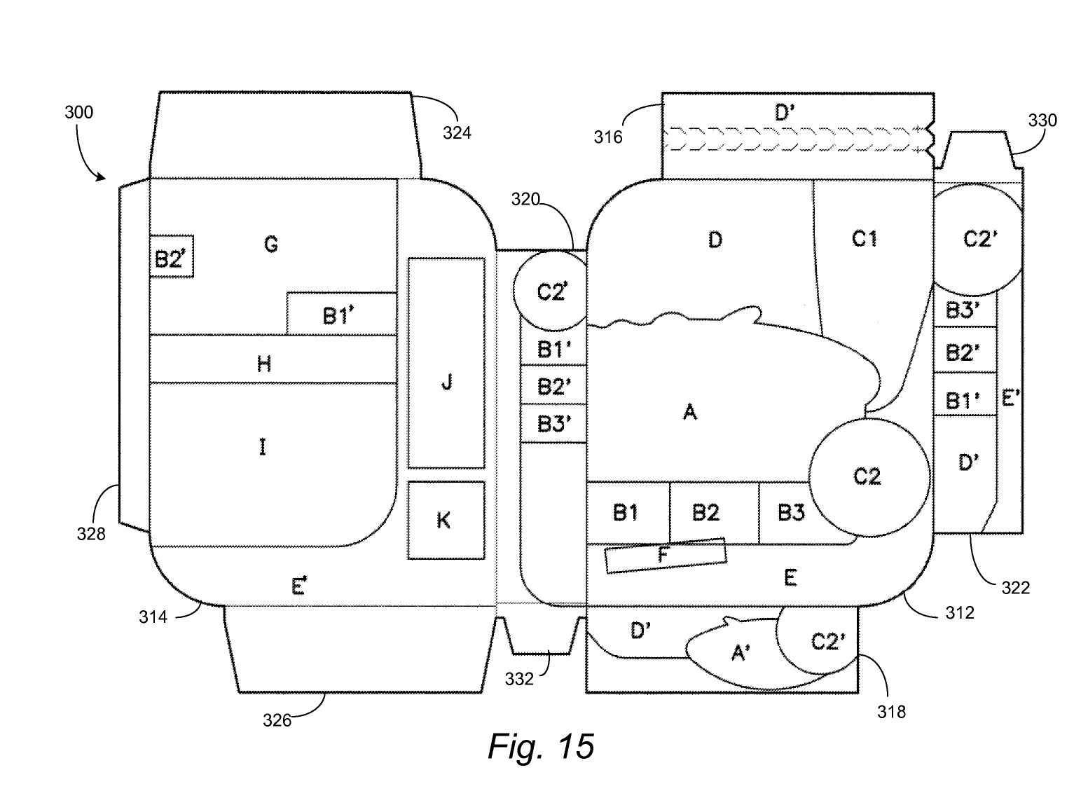

Sleeve 300 can also include first closed corner 306 and second closed corner 308. First closed corner 306 and second closed corner 308 can provide a flat base for packaging assembly 100 when packaging assembly 100 is positioned in an upright position on a display shelf at the point-of-sale. Also, as more fully set forth below, sleeve 300 includes sleeve front 312, sleeve back 314 (see FIGS. 14 and 15), sleeve first side 316, sleeve second side 318 (see FIGS. 14 and 15), sleeve third side 320, and sleeve fourth side 322 (see FIGS. 14 and 15).

Opening feature 310 can be located along sleeve first side 316. Opening feature 310 can include any type of structure for opening sleeve 300. For example opening feature 310 can include a perforated opening feature, a resealable opening feature, a tear-away opening feature, a tongue-and-groove opening feature, and combinations thereof. In one aspect, opening feature 310 can include a perforated opening strip. When a consumer desires removing container 200 from sleeve 300, opening feature 310 is navigated to allow access to container 200.

FIG. 1 depicts packaging assembly 100 in an upright position. Even though packaging assembly 100 can be stacked and/or positioned at the point-of-sale in any manner, it is contemplated that the upright position maximizes the viewable surface of sleeve 300 when a potential consumer views packaging assembly 100 at the point-of-sale. As more fully set forth below, the maximization of the viewable surface facilitates the communication of the predetermined message at the point-of-sale.

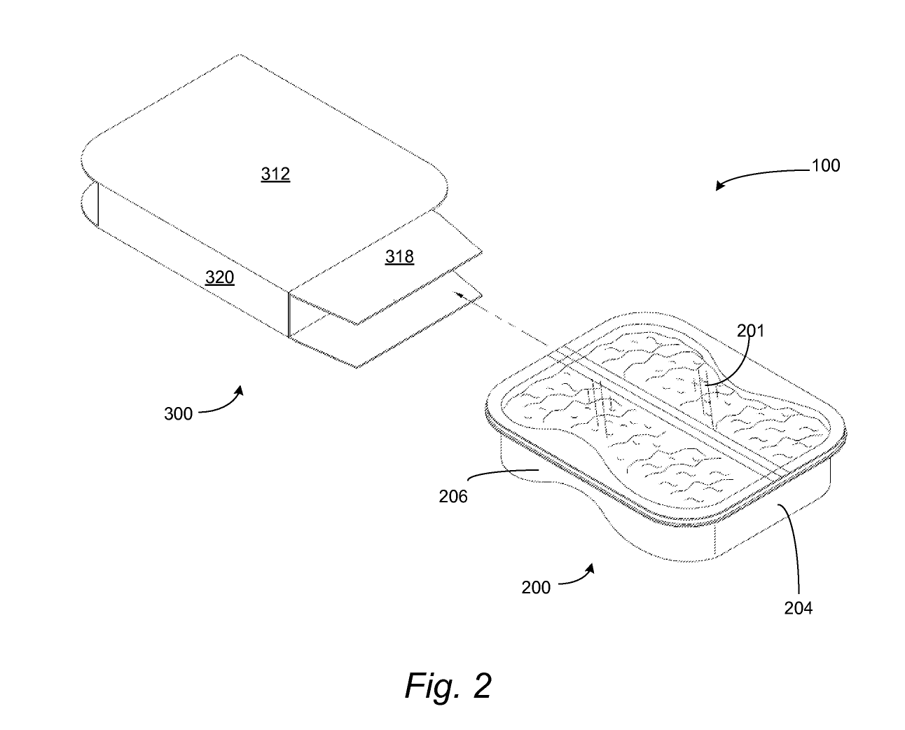

FIG. 2 is an example perspective of packaging assembly 100 of FIG. 1 that illustrates packaging assembly 100 prior to the sealing of sleeve 300. As indicated in FIG. 2, container 200 can optionally include removable film 201. Removable film 201 can seal a consumable product in container 200. Removable film 201 can include a venting and/or steaming perforation associated with removable film 201 to regulate steaming during a cooking operation. Container 200 is configured to maintain a variety of food products including room temperature food products, refrigerated food products, food products and the like. In one aspect, the food product in container 200 is a prepared meal that includes one or more vegetables, fruits, proteins, starches, spices, peppers, sauces and combinations thereof.

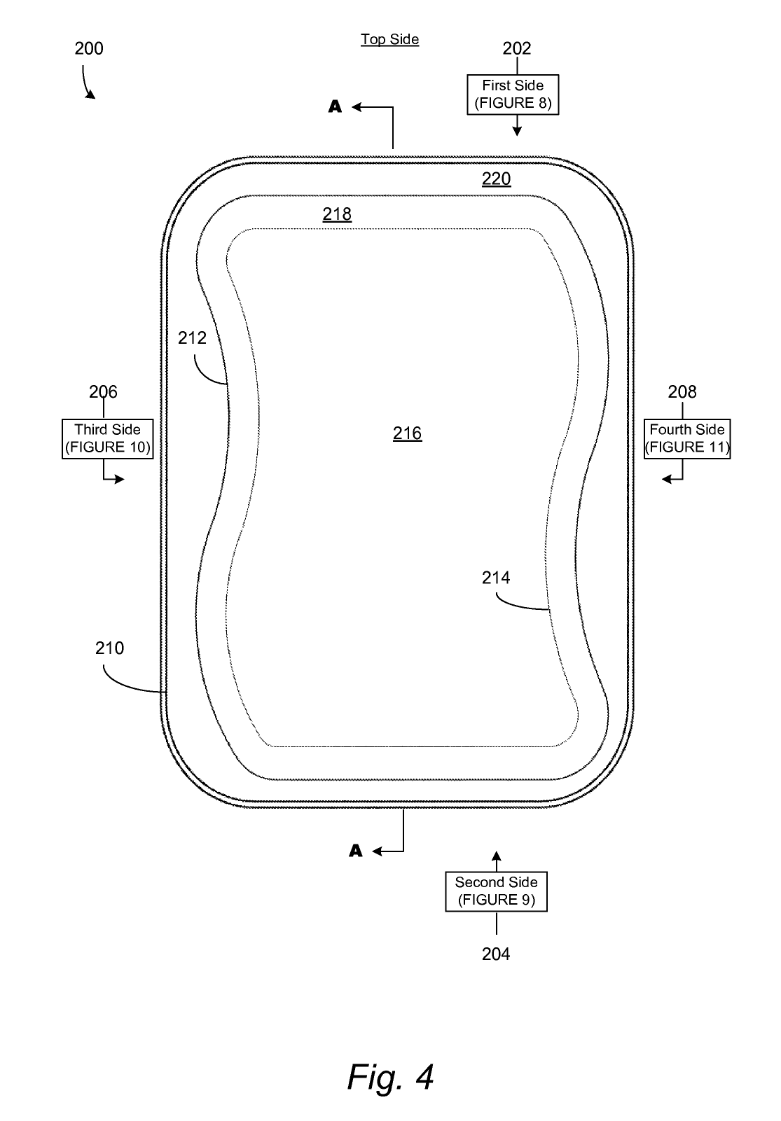

During packaging, container first side 202 (see FIG. 4) is first slid into sleeve 300 so that container first side 202 is substantially adjacent sleeve first side 316. Sleeve second side 318 is then sealed in a conventional manner.

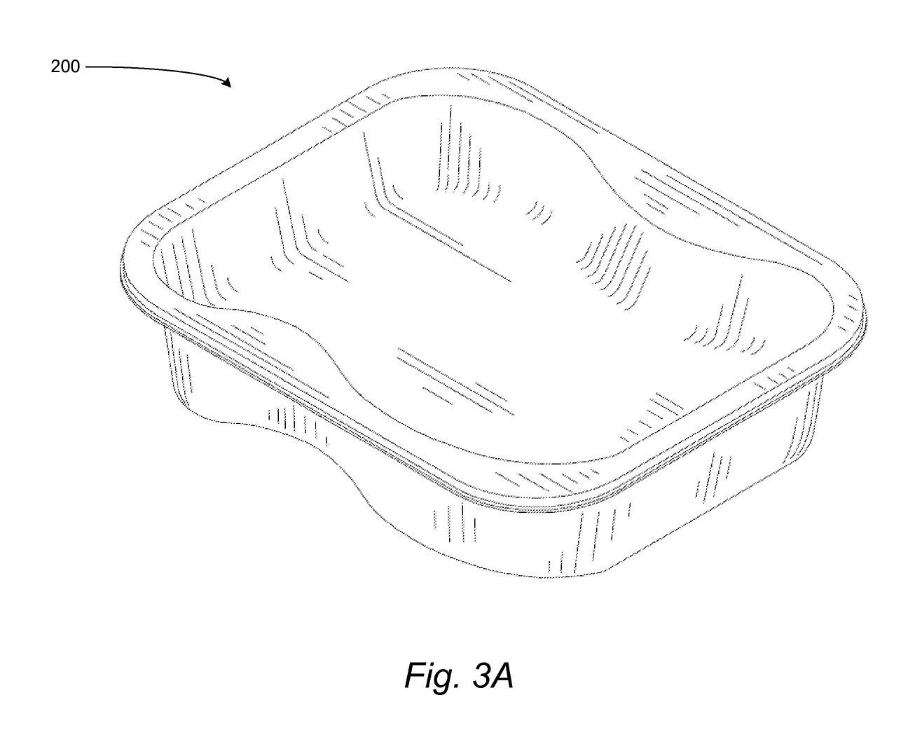

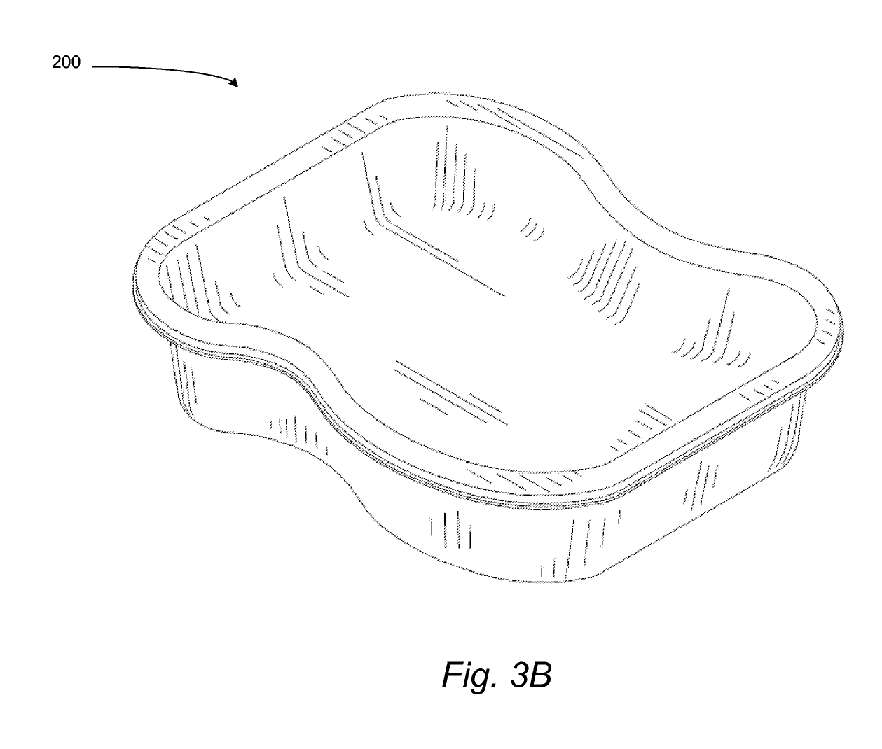

As more fully set forth herein, the Detailed Description is divided into a section related to container 200 and a section related to sleeve 300. FIGS. 3-13 are indicated herein to describe several features associated with container 200 and FIGS. 14-21 are indicted herein to describe several features associated with sleeve 300. Even though several examples are set forth throughout the Detailed Description, the Detailed Description further includes a section describing several examples associated with the nexus formed by various combinations of features of container 200 and sleeve 300 in order to facilitate a predetermined message and drive a consumer impression.

Container

FIG. 3A is an example perspective of container 200 of packaging assembly 100. FIG. 3A is depicted herein to further exemplify the structural features discussed in FIGS. 4-13. FIG. 3A can be referenced in combination with the discussion set forth below in association with FIGS. 4-13 to more fully appreciate many of the structural features set forth in relation to container 200. FIG. 3B is another embodiment of container 200. Even though FIG. 3A is referenced below with respect to the features of packaging assembly 100, it will be evident from the disclosure herein that similar features between FIGS. 3A and 3B can result in a similar nexus for driving a predetermined message.

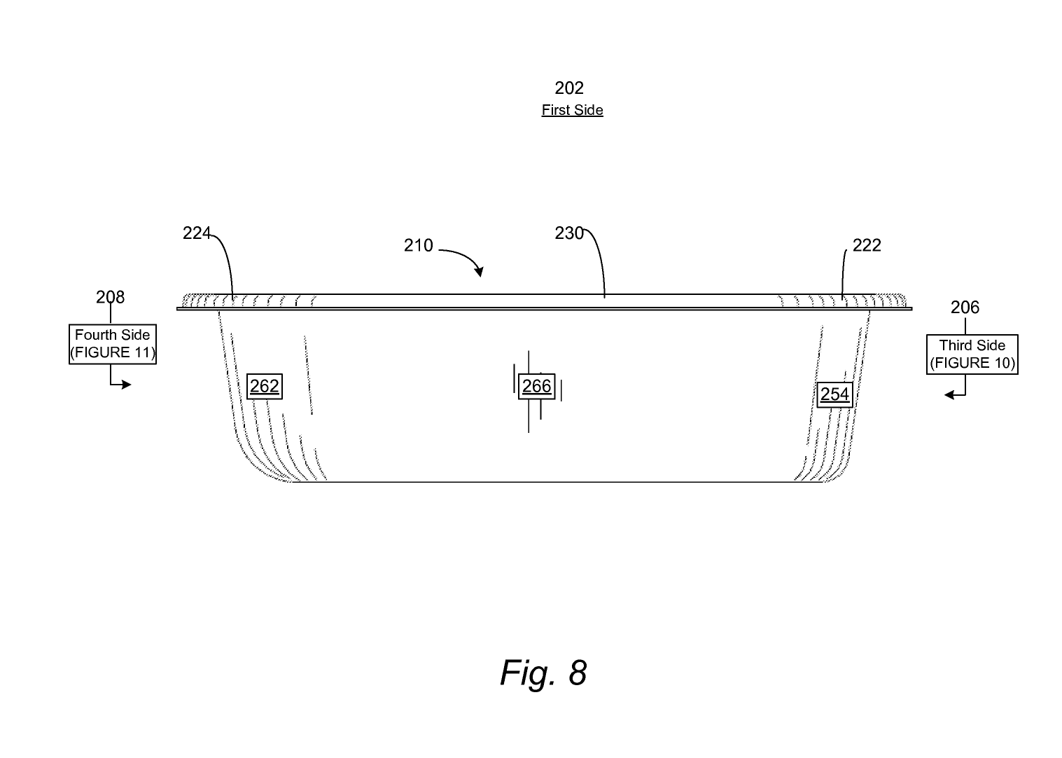

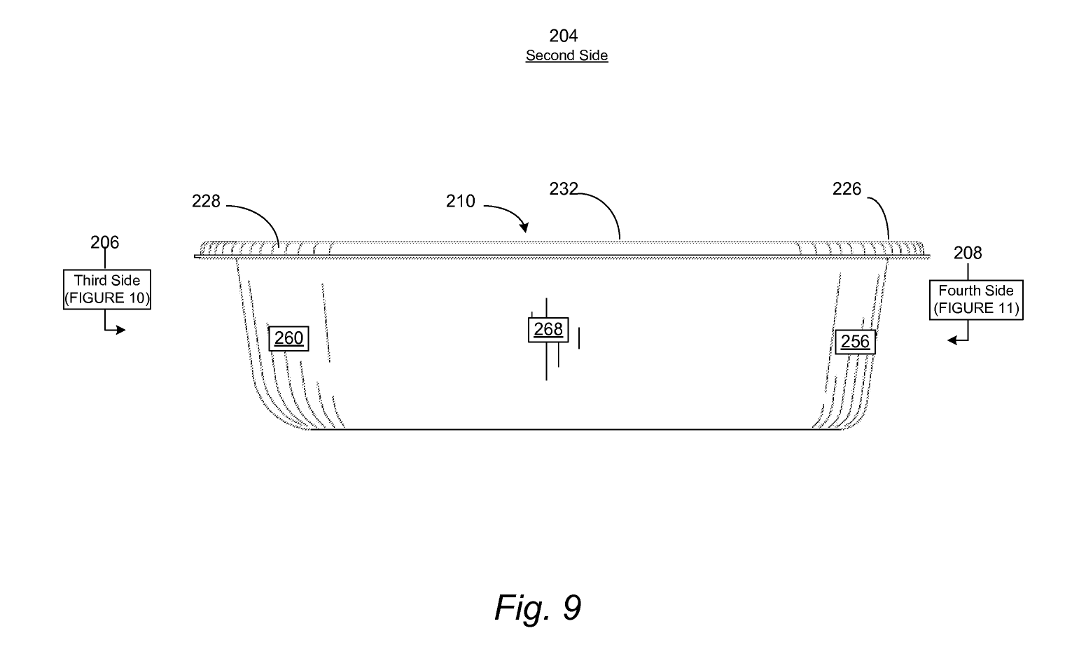

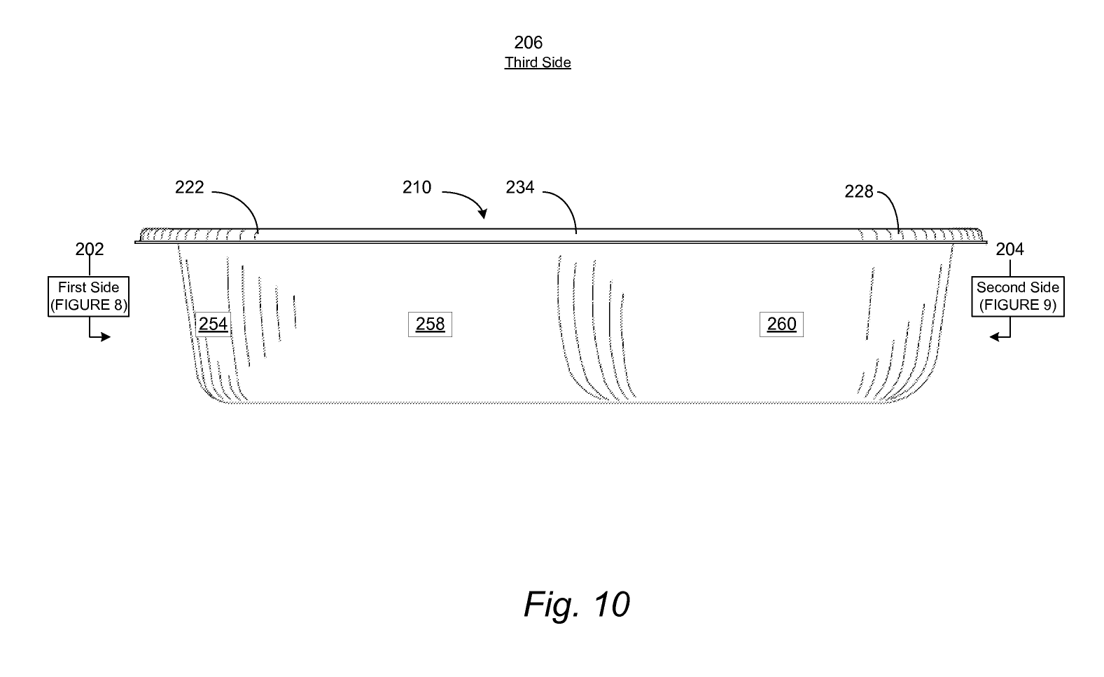

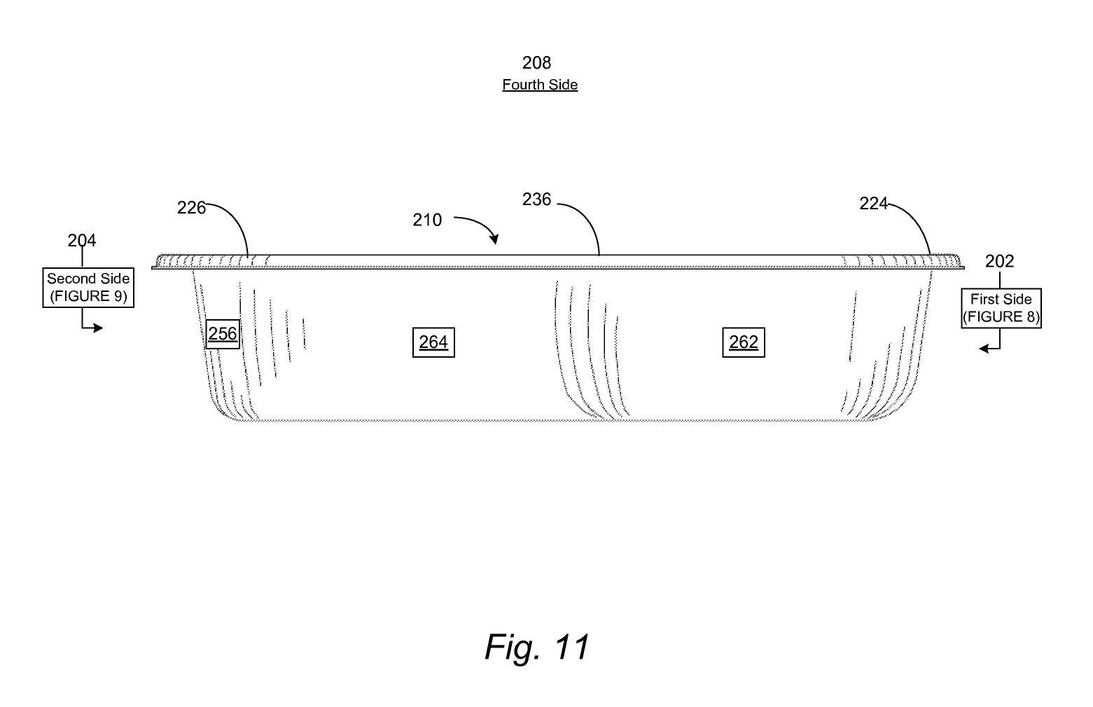

FIG. 4 is an example top side of container 200 of packaging assembly 100. Container 200 includes container first side 202 which is more fully set forth in FIG. 8, container second side 204 which is more fully set forth in FIG. 9, container third side 206 which is more fully set forth in FIG. 10, and container fourth side 208 which is more fully set forth in FIG. 11.

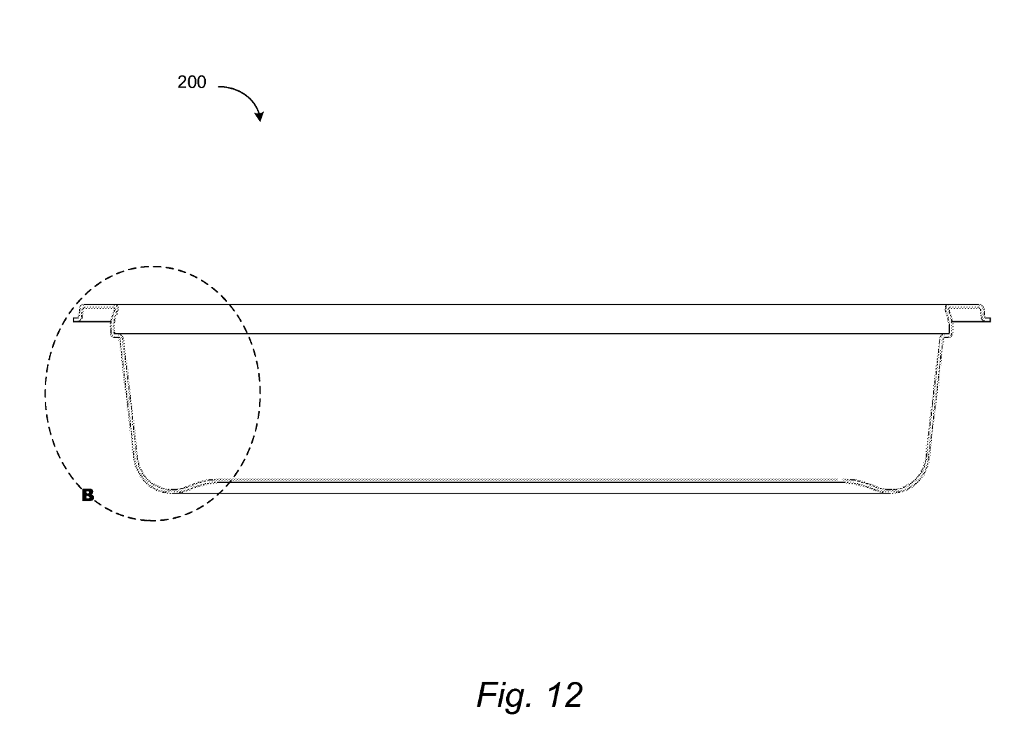

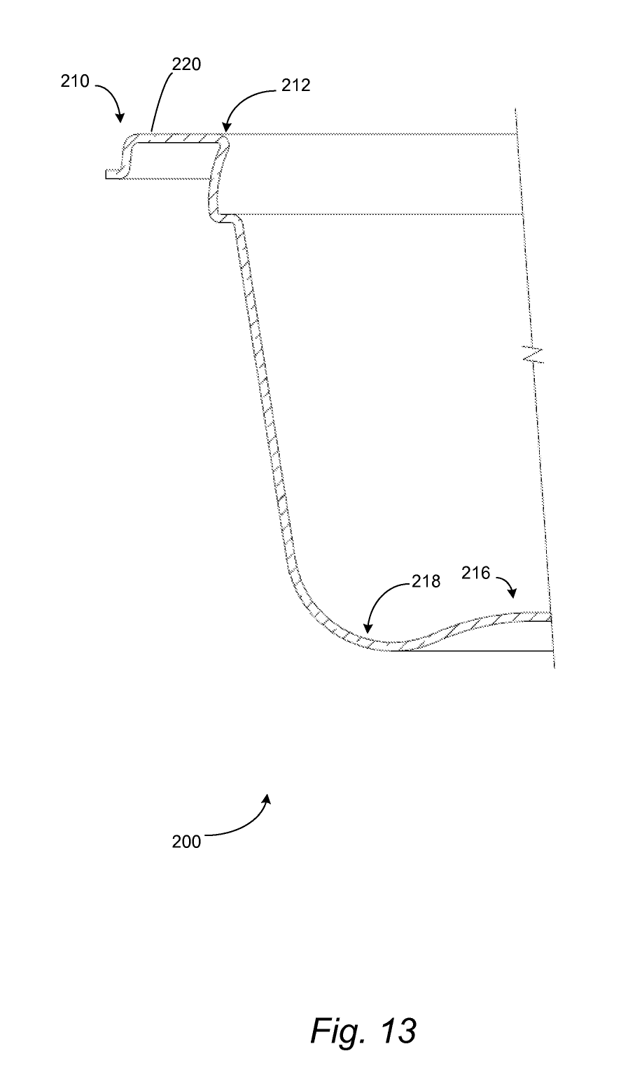

Container 200 includes container rim surface 220 having rim outer edge 210 and rim inner edge 212. Container rim surface 220 can include a surface area sufficient for adhering removable film 201 to container rim surface 220. Also, the container rim surface 220 can provide an area for a consumer to grasp container 200 and position in and/or remove container 200 from a cooking device. FIG. 13 includes a magnified view of the cross section indicated in FIG. 12 along lines A-A of FIG. 4. Rim outer edge 210 can be the end of container rim surface 220. Also, as indicated in FIG. 13, rim outer edge 210 can include a reverse “L-shaped” lip extending downwardly from container rim surface 220. Rim inner edge 212 can include a curve or bend to transition rim inner edge 212 to a wall of container 200. In other aspects, rim inner edge 212 can include an angular edge as the transition from container rim 220 to a wall of container 200.

Referring back to FIG. 4, container 200 also includes container bottom 216. Container bottom 216 can be generally flat and can end at bottom edge 214. Bottom edge 214 can include a portion of the container where the container bottom ends and channel transition 218 begins. As further indicated in FIG. 13, channel transition 218 can include a curve and/or bend that structurally transitions the generally flat container bottom 216 to a wall of container 200. Channel transition 218 can transition downwardly from container bottom 216 so that container bottom 216 is orientated above a channel low point of channel transition 218. As such, when container 200 is positioned on a surface, channel transition 218 engages the surface and container bottom 216 can be offset from the surface. Yet, it is contemplated the channel transition 218 is not incorporated with container 200.

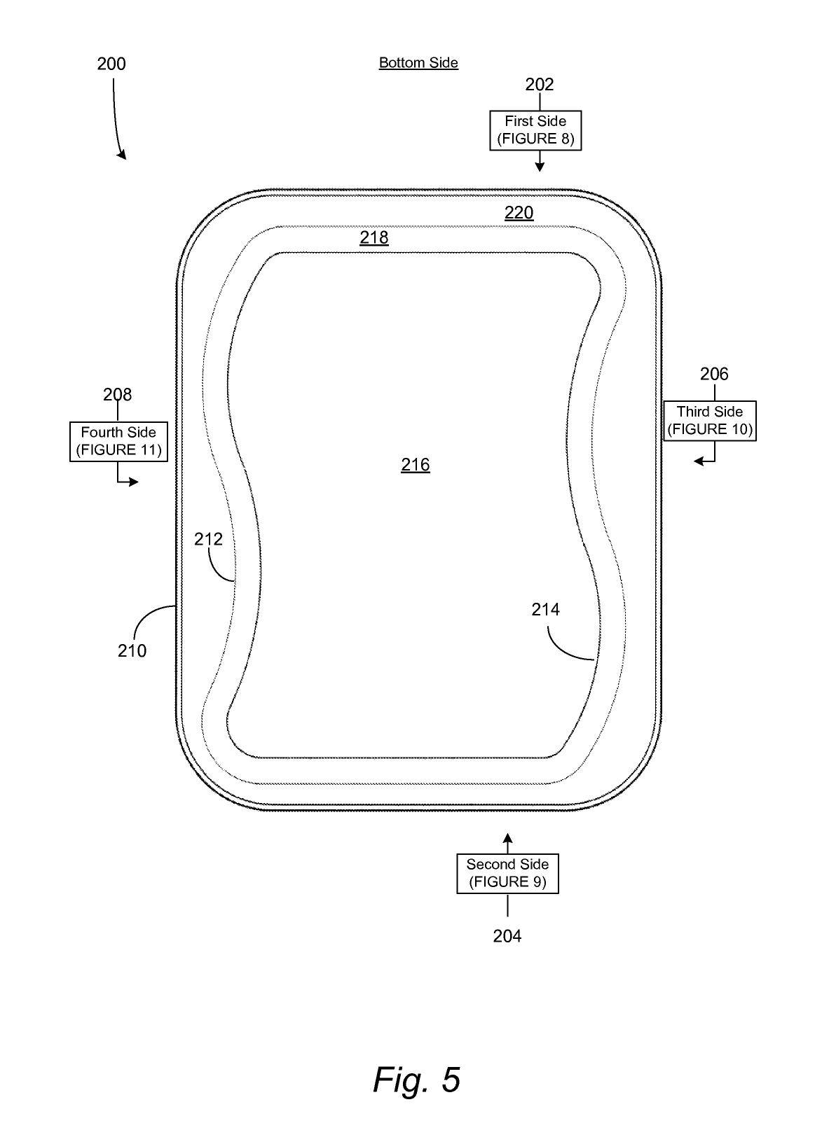

FIG. 5 is an example bottom side of container 200 of packaging assembly 100. In comparison to FIG. 3, FIG. 5 depicts container 200 with the top side facing downwardly.

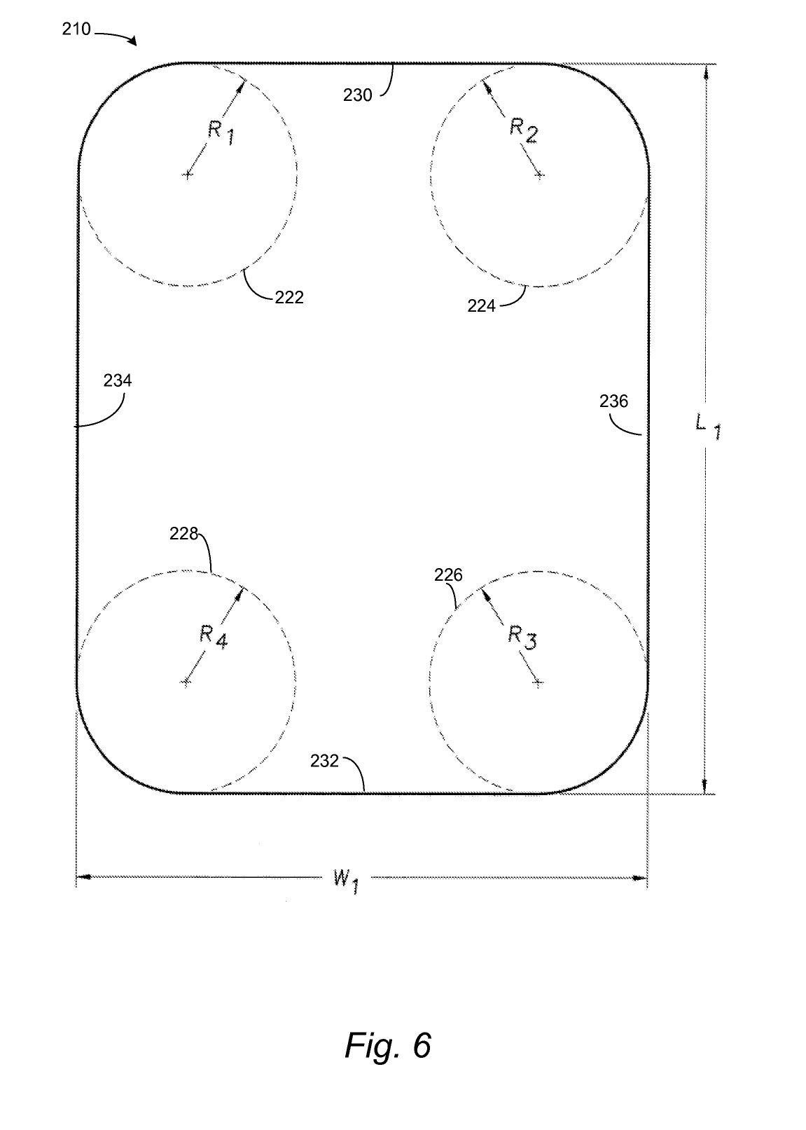

FIG. 6 is an example dimensional diagram of rim outer edge 210 of the container 200. For ease of description, rim outer edge 210 is described in FIG. 6 apart from any depiction of other attributes of container 200. Rim outer edge 210 includes a length L₁ and a width W₁. Length L₁ and width W₁ can include any length and/or width. Yet, in one aspect, L₁ is about 6.5 inches to about 7.5 inches. Width W₁ can be about 5.0 inches to about 6.0 inches. Length L₁ and width W₁ can be further described as having an aspect ratio. For example, the aspect ratio of Length L₁ to width W₁ can be about 1.25. As another example, the aspect ratio of Length L₁ to width W₁ can be about 1 to about 2. Given the aspect ratio, the length L₁ and the width W₁ can be any size while maintaining the aspect ratio. Yet, other dimensions beyond any aspect ratio discussed herein are contemplated as initially stated above.

Rim outer edge 210 of container 200 includes first side outer rim edge 230, second side outer rim edge 232, third side outer rim edge 234, and fourth side outer rim edge 236. First side outer rim edge 230 is generally parallel to second side outer rim edge 232 and generally perpendicular to third side outer rim edge 234 and fourth side outer rim edge 236. Second side outer rim edge 232 is generally parallel to first side outer rim edge 230 and generally perpendicular to third side outer rim edge 234 and fourth side outer rim edge 236. Third side outer rim edge 234 is generally parallel to fourth side outer rim edge 236 and generally perpendicular to first side outer rim edge 230 and second side outer rim edge 232. Fourth side outer rim edge 236 is generally parallel to third side outer rim edge 234 and generally perpendicular to first side outer rim edge 230 and second side outer rim edge 232.

Third side outer rim edge 234 structurally transitions to first side outer rim edge 230 via first corner curve 222. First corner curve 222 can be formed from a portion of an ellipse having a radius R₁. In one aspect, the ellipse is a circle. In another aspect, third side outer rim edge 234 and first side outer rim edge 230 are tangent to a circumference of the ellipse formed by R₁.

First side outer rim edge 230 structurally transitions to fourth side outer rim edge 236 via second corner curve 224. Second corner curve 224 is formed from a portion of an ellipse having a radius R₂. In one aspect, the ellipse is a circle. In another aspect, first side outer rim edge 230 and fourth side outer rim edge 236 are tangent to a circumference of the ellipse formed by R₂.

Fourth side outer rim edge 236 structurally transitions to second side outer rim edge 232 via third corner curve 226. Third corner curve 226 is formed from a portion of an ellipse having a radius R₃. In one aspect, the ellipse is a circle. In another aspect, fourth side outer rim edge 236 and second side outer rim edge 232 are tangent to a circumference of the ellipse formed by R₃.

Second side outer rim edge 232 structurally transitions to third side outer rim edge 234 via fourth corner curve 228. Fourth corner curve 228 is formed from a portion of an ellipse having a radius R₄. In one aspect, the ellipse is a circle. In another aspect, second side outer rim edge 232 and third side outer rim edge 234 are tangent to a circumference of the ellipse formed by R₄.

As indicated in FIG. 6, R₁, R₂, R₃, and R₄ can have the same length. Accordingly, the curves associated with the respective radius can be generally the same. R₁, R₂, R₃, and R₄ can have the same length within a threshold. For example, the threshold can be plus or minus about 0.1 inches. As another example, R₁, R₂, R₃, and R₄ can be about 0.5 inches to about 1.5 inches. Yet, the dimensions related to R₁, R₂, R₃, and R₄ are not limited to the dimensions indicated herein. As indicated above W₁ and L₁ of rim outer edge 210 can vary. Rim outer edge 210 can also be associated with an aspect ratio. Accordingly, a manufacturer that desires to fabricate a container that is larger or small than the dimensions indicated herein could maintain aspect ratios between R₁, R₂, R₃, and R₄ and one or more of L₁ and/or W₁ to change the overall size of container 200 without optionally affecting the shape. As more fully set forth below, first corner curve 222 and third corner curve 226 create a nexus with first corner opening 302 of sleeve 300 and second corner opening 304 of sleeve 300, respectively.

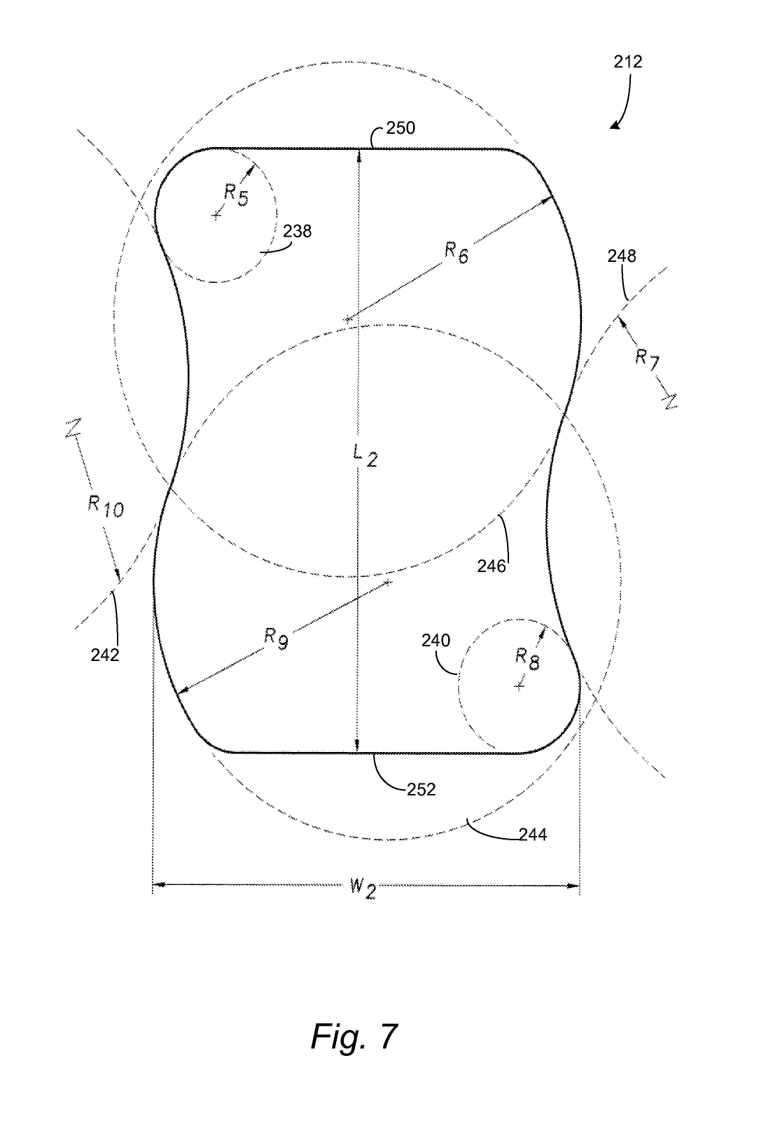

FIG. 7 is an example dimensional diagram of rim inner edge 212 of the container 200. For ease of description, rim inner edge 212 is described in FIG. 7 apart from any depiction of other attributes of container 200. Rim inner edge 212 includes a length L₂ and a width W₂. Length L₂ and width W₂ can include any length or width. Yet, in one aspect, L₂ is about 6.0 inches to about 7.0 inches. Width W₂ can be about 4.5 inches to about 5.5 inches. Length L₂ and width W₂ can be further described as having an aspect ratio. For example, the aspect ratio of Length L₂ to width W₂ can be about 1.25. As another example, the aspect ratio of Length L₂ to width W₂ can be about 1 to about 2. Given the aspect ratio, the length L₂ and the width W₂ can be any size while maintaining the aspect ratio. Yet, other dimensions beyond any aspect ratio discussed herein are contemplated as initially stated above.

Rim inner edge 212 of container 200 includes first side inner rim edge 250 and second side inner rim edge 252. First side inner rim edge 250 is generally parallel to second side inner rim edge 252.

As used herein, the term “convex” refers to a curve opening inwardly toward a centerline along the length L₂ of container 200. As used herein, the term “concave” refers to a curve opening outwardly away from a centerline along the length L₂ of container 200. Rim inner edge 212 includes fourth side convex curve 246. Fourth side convex curve 246 is formed from a portion of an ellipse having a radius R₆. In one aspect, the ellipse can be a circle. In another aspect, a line formed from first side inner rim edge 250 is secant to a circumference of an ellipse formed from R₆.

Fourth side convex curve 246 structurally transitions to fourth side concave curve 248. Fourth side concave curve 248 is formed from a portion of an ellipse having a radius R₇. In one aspect, the ellipse is a circle. In another aspect, a line formed from second side inner rim edge 252 is secant to a circumference of an ellipse formed from R₇. In yet another aspect, a circumference of the ellipse formed from R₇ is tangent to a circumference of the ellipse formed from R₆.

Fourth side concave curve 248 structurally transitions to second corner curve 240. Second corner curve 240 is formed from a portion of an ellipse having a radius R₈. In one aspect the ellipse is a circle. In another aspect, a circumference of an ellipse formed from R₇ and a line formed from second side inner rim edge 252 are tangent to a circumference of an ellipse formed from R₈.

Second corner curve 240 structurally transitions to second side inner rim edge 252. Fourth side convex curve 246, fourth side concave curve 248 and second corner curve 240 form the fourth side of the rim inner edge 212.

Inner rim edge 252 transitions to third side convex curve 244. Third side convex curve 244 is formed from a portion of an ellipse having a radius R₉. In one aspect, the ellipse is a circle. In another aspect, a line formed from second side inner rim edge 252 is secant to a circumference of an ellipse formed from R₉.

Third side convex curve 244 structurally transitions to third side concave curve 242. Third side concave curve 242 is formed from a portion of an ellipse having a radius R₁₀. In one aspect, the ellipse is a circle. In another aspect, a line formed from first side inner rim edge 250 is secant to a circumference of an ellipse formed from R₁₀. In yet another aspect, a circumference of the ellipse formed from R₁₀ is tangent to a circumference of the ellipse formed from R₉.

Third side concave curve 242 geometrically transitions to first corner curve 238. First corner curve 238 is formed from a portion of an ellipse having a radius R₅. In one aspect, the ellipse is a circle. In another aspect, a circumference of an ellipse formed from R₁₀ and a line formed by first side inner rim edge 250 are tangent to a circumference of an ellipse formed from R₅.

Third side convex curve 244, third side concave curve 242 and first corner curve 238 form the third side of the rim inner edge 212. As indicated in FIG. 7, first corner curve 238 and second corner curve 240 are generally diagonally oriented with respect to one another. Also, fourth side convex curve 246 and third side convex curve 244 are generally diagonally oriented with respect to one another. Likewise, fourth side concave curve 248 and third side concave curve 242 are generally diagonally oriented with respect to one another.

As further indicated in FIG. 7, R₅ and R₈ can generally have the same length. R₅ and R₈ can also have the same length within a threshold. For example, the threshold can be plus or minus about 0.1 inches. As an example, R₅ and R₈ can be about 0.5 inches to about 0.8 inches. In the situation where R₅ and R₈ are generally the same, first corner curve 238 and second corner curve 240 can generally be the same curve.

R₆ and R₉ can generally have the same length. R₆ and R₉ can also have the same length within a threshold. For example, the threshold can be plus or minus about 0.1 inches. For example, R₆ and R₉ can be about 5.0 inches to about 6.0 inches. In the situation where R₆ and R₉ are generally the same, fourth side convex curve 246 and third side convex curve 244 are generally the same curve.

Likewise, R₇ and R₁₀ can generally have the same length. R₇ and R₁₀ can also have the same length within a threshold. For example, the threshold can be plus or minus about 0.1 inches. As another example, R₇ and R₁₀ can be about 2.5 inches to about 3.5 inches. In the situation where, R₇ and R₁₀ are generally the same, fourth side concave curve 248 and third side concave curve 242 are generally the same curve.

As another example combination: R₅ and R₈ can be generally the same length, R6 and R₉ can be generally the same length, R₇ and R₁₀ can be generally the same length, the length of R₆ and R₉ can be greater than the length of R₇ and R₁₀, and the length of R₆, R₇, R₉ and R₁₀ can be greater than the length of R₅ and R₈.

As indicated in FIG. 13, rim inner edge 212 transitions downwardly to form the walls of container 200. The container walls can be offset from vertical so that the walls slope inwardly from top to bottom. Accordingly, the container walls can include the same general shape as rim inner edge 212 as depicted in FIG. 3A and FIG. 3B. As further indicated in FIG. 3A and FIG. 3B, the structure of rim inner edge 212 and the respective walls form a geometric cooking indicator. For example, as indicated in FIGS. 3A and 3B, the container shape is formed to indicate a steam vapor, which can convey a predetermined message related to a steam cooking operation used to cook the food product located in container 200 for consumption. The purpose of the geometric cooking indicator is more fully set forth below. Even though the examples herein relate to a geometric cooking indicator that represents a steam vapor, other geometric cooking indicators are contemplated. For example, a geometric cooking indicator can be structured to indicate a water droplet in order to represent a boil cooking operation. As another example, a geometric cooking indicator can be structured to indicate a flame to represent a grill cooking operation. As yet another example, a geometric cooking indicator can be structure to indicate a heat wave to represent a conventional oven cooking operation. As more fully set forth below, the steam indicator can create a nexus between container 200 and sleeve 300 to facilitate a predetermined message and drive a consumer impression.

FIG. 8 is an example container first side 202 of container 200 of packaging assembly 100. Container first side 202 includes a view of rim outer edge 210 having first corner curve 222, second corner curve 224, and first side outer rim edge 230. Container first side 202 also includes fourth side convex curve wall 262 which extends downwardly from and is generally shaped to conform to fourth side convex curve 246. Container first side 202 further includes first side inner rim edge wall 266 which extends downwardly from and generally shaped to conform to first side inner rim edge 250. Container first side 202 also includes first corner curve wall 254 which extends downwardly from and generally shaped to conform to first corner curve 238.

FIG. 9 is an example container second side 204 of container 200 of packaging assembly 100. Container second side 204 includes a view of rim outer edge 210 having third corner curve 226, fourth corner curve 228, and second side outer rim edge 232. Container second side 204 also includes third side convex curve wall 260 which extends downwardly from and generally shaped to conform to third side convex curve 244. Container second side 204 further includes second side inner rim edge wall 268, which extends downwardly from and generally shaped to conform to second side inner rim edge 252. Container second side 204 also includes second corner curve wall 256 which extends downwardly from and generally shaped to conform to second corner curve 240.

FIG. 10 is an example container third side 206 of container 200 of packaging assembly 100. Container third side 206 includes a view of rim outer edge 210 having fourth corner curve 228, first corner curve 222, and third side outer rim edge 234. Container third side 206 also includes first corner curve wall 254 which extends downwardly from and generally shaped to conform to first corner curve 238. Container third side 206 further includes third side concave curve wall 258 which extends downwardly from and generally shaped to conform to third side concave curve 242. Container third side 206 also includes third side convex curve wall 260 which extends downwardly from and generally shaped to conform to third side convex curve 244.

FIG. 11 is an example container fourth side 208 of container 200 of the packaging assembly 100. Container fourth side 208 includes a view of rim outer edge 210 having second corner curve 224, third corner curve 226, and fourth side outer rim edge 236. Container fourth side 208 also includes second corner curve wall 256 which extends downwardly from and generally shaped to conform to second corner curve 240. Container fourth side 208 further includes fourth side concave curve wall 264 which extends downwardly from and generally shaped to conform to fourth side concave curve 248. Container fourth side 208 also includes fourth side convex curve wall 262 which extends downwardly from and generally shaped to conform to fourth side convex curve 246.

Sleeve

FIG. 14 is an example structural diagram of sleeve 300 of packaging assembly 100. Sleeve 300 includes sleeve front 312, sleeve back 314, sleeve first side 316, sleeve second side 318, sleeve third side 320, and sleeve fourth side 322. Sleeve 300 further includes a plurality of sealing panels. For example, sleeve 300 can include first back sealing panel 324, second back sealing panel 326, third back sealing panel 328, fourth side sealing panel 330, and third side sealing panel 332. To form the sleeve as indicated in FIG. 1, sleeve first side 316 overlays first back sealing panel 324 and fourth side sealing panel 330. Sleeve second side 318 overlays second back sealing panel 326 and third side sealing panel 332. Sleeve fourth side 322 overlays third back sealing panel 328. As such, a formed sleeve 300 is configured as indicated in FIGS. 1 and 2. As indicated in FIG. 1, packaging assembly 100 is arranged so that container top side is adjacent sleeve front 312, container bottom side is adjacent sleeve back 314, container first side 202 is adjacent sleeve first side 316, container second side 204 is adjacent sleeve second side 318, container third side 206 is adjacent sleeve third side 320, and container fourth side 208 is adjacent sleeve fourth side 322.

FIG. 14 includes first front curve 334, second front curve 336, first back curve 338, and second back curve 340. As indicated in FIG. 14, the curves associated with R₁₁, R₁₂, R₁₃, and R₁₄ can be formed from an ellipse having the same length. Also, the curves associated with R₁₁, R₁₂, R₁₃, and R₁₄ can be formed from an ellipse having the same radius within a threshold. For example, the threshold can be plus or minus about 0.1 inches. As another example, R₁₁, R₁₂, R₁₃, and R₁₄ can be about 0.5 inches to about 1.5 inches. In comparison between FIG. 14 and FIG. 6, R₁₁ and R₁₂ can be the same length as R₁ or the same length within a threshold. In another example, R₁₃ and R₁₄ can be the same length as R₃. In still another example, R₁, R₂, R₃, R₄, R₁₁, R₁₂, R₁₃, and R₁₄ can be the same length. As such, when sleeve 300 is formed as depicted in FIG. 1, first front curve 334 and first back curve 338 form first corner opening 302. Second front curve 336 and second back curve 340 form second corner opening 304. As indicated in FIG. 1, the formed sleeve 300 also includes first closed corner 306 and second closed corner 308.

When container 200 is positioned in sleeve 300, first corner curve 222 of rim outer edge 210 geometrically aligns (e.g., within the same curved plane) with first front curve 334 and first back curve 338 of sleeve 300 to cause a nexus between sleeve 300 and container 200 at first corner opening 302. Likewise, when container 200 is positioned in sleeve 300, third corner curve 226 of rim outer edge 210 geometrically aligns (e.g., within the same curved plane) with second front curve 336 and second back curve 340 of sleeve 300 to cause a nexus between sleeve 300 and container 200 at second corner opening 304. As such, packaging assembly 100 is stabilized in an upright position via first closed corner 306 and/or second closed corner 308 and a potential consumer can readily view container 200 and the food product within container 200 via first corner opening 302 and/or second corner opening 304. Moreover, the aforementioned nexus at first corner opening 302 and second corner opening 304 facilitates a geometric tie between container 200 and sleeve 300. As such, a consumer who can only recall the configuration of container 200 has an indicator to associate with sleeve 300 upon making a subsequent purchase of the product associated with packaging assembly 100.

FIG. 15 is an example indicator field diagram associated with sleeve 300 of packaging assembly 100. In addition to the structural indicia associated with packaging assembly 100 discussed above, sleeve 300 further includes a plurality of indicator fields for further driving a nexus between the components of packaging assembly 100 and ultimately indicating a predetermined message for a consumer impression. As more fully set forth below, the arrangement of the indicator fields in combination with the structural indicia of packaging assembly 100 collectively signal and/or reinforce a predetermined message for causing a consumer impression.

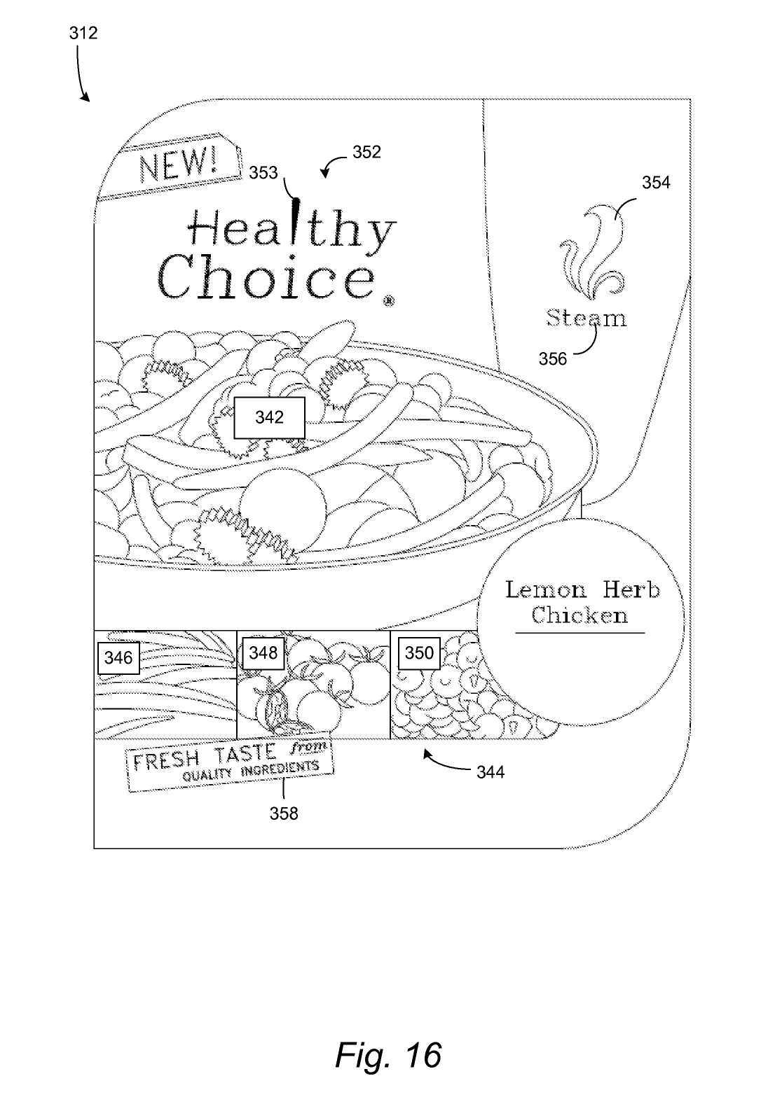

Sleeve front 312 includes prepared product indictor field (A). Prepared product indicator field (A) is arranged in coordination with the plurality of fields to provide a primary field for prepared product indicia 342 on sleeve front 312. Prepared product indicia 342 signals a cooked state of the product located in container 200 just prior to use and/or consumption. As an example associated with FIG. 16, prepared product indicia 342 includes an image of a microwave cooked product including a plurality of steamed ingredients and signals a prepared state of the food product located in container 200. As such, the arrangement of prepared product indicator field (A) and prepared product indicia 342: signal a primary view of the state of the cooked food product when packaging assembly 100 is positioned in an upright position at a retail outlet; signal a product tie between the food product located in container 200 and a state of the food product after the food product has been cooked; and/or signal a type of food product located in container 200.

Ingredient indicator field (B) is arranged in coordination with the plurality of fields to tie to prepared product indicator field (A) and a food product in container 200. Ingredient indicator field (B) can include ingredient indicator sub-fields (B1)-(B3) as an array of ingredients of the food product in container 200. As indicated in FIG. 15, ingredient indicator field (B) is arranged adjacent prepared product indicator field (A) to provide a nexus between two different product states (e.g., the origin state of the ingredient and the cooked state of the product). In one aspect, sub-fields (B1)-(B3) are arranged below the prepared product indicator field (A). For example, as indicated in FIG. 16, ingredient origin indicia 344 as populated in ingredient indicator field (B) can include first ingredient origin indicia 346, second ingredient origin indicia 348, and third ingredient origin indicia 350. Again, first ingredient origin indicia 346, second ingredient origin indicia 348, and third ingredient origin indicia 350 can include an array associated with ingredients indicated in prepared product indicia 342 and the food product located in container 200.

First ingredient origin indicia 346 can include an image of at least one ingredient at its origin. For example, the image can include an image of the at least one ingredient in a prepackaging state. As another example, the image can include an image of the at least one ingredient in a natural state to create an impression of freshness. Stated another way, first ingredient origin indicia 346 includes an image that creates a freshness impression of at least one ingredient of prepared product indicia 342 that indicates the ingredient in a cooked state. Likewise, second ingredient origin indicia 348 and third ingredient origin indicia can include respective images associated with prepared product indicia 342. Ingredient origin indicia 344 facilitates a nexus for indicating an impression of “freshness” or “market fresh” ingredients in association with prepared product indicia 342 and the food product located in container 200.

As an example associated with FIG. 16, container 200 can include a food product that includes green beans as an ingredient of a food product in a packaged state. Prepared product indicia 342 indicates the green beans in a cooked state and first ingredient origin indicia 344 indicates the green beans in a prepackaged state. As another example associated with FIG. 16, container 200 can include a food product that includes tomatoes as an ingredient of a food product in a packaged state. Prepared product indicia 342 indicates the tomatoes in a cooked state and second ingredient origin indicia 348 indicates the tomatoes in a prepackaged state. As yet another example associated with FIG. 16, container 200 can include a food product that includes a starch as an ingredient of a food product in a packaged state. Prepared product indicia 342 indicates the starch in a cooked state and third ingredient origin indicia 350 indicates the starch in a prepackaged state. As such, ingredient indicator field (B) and ingredient origin indicia 344: signal a nexus between the cooked state indicated by prepared product indicator 342 and an origin state of the ingredients; signal a nexus between the state of the food product in container 200 and the origin state of the ingredients; and/or drive a predetermined message that indicates a perception of freshness of the ingredients.

Brand tying indicator field (C) can include subfields (C1) and (C2). Brand tying indicator field (C) is structured and arranged in coordination with the plurality of fields to tie to brand indicator field (D). As an example associated with FIG. 16, brand indicator field (D) can be populated with brand indicia 352. In this example, brand indicia 352 includes the textual indicia Healthy Choice® along with textual indicia 353 indicated by the exclamation point. As indicated in FIG. 16, brand tying indicator field (C) is shaped to resemble at least one portion of brand indicia 352. In one aspect, brand tying indicator field (C) is shaped to resemble textual indicia 353. As such, indicia associated with subfields (C1) and (C2) are tied to brand indicia 352 via the structural coordination between brand tying indicator field (C) and brand indicia 352.

Subfield (C1) can include cooking method indicia. As an example associated with FIG. 16, the cooking method indicia can include steam image indicia 354 and/or steam textual indicia 356. As indicated, steam image indicia 354 includes a steam vapor and steam textual indicia 356 includes the text “Steam”. The cooking method indicia creates a nexus between prepared product indicia 342 and the cooking method indicia. For example, the cooking method indicia in subfield (C1) indicates steam cooking. The steam cooking indication ties to prepared product indicia 342 by indicating that the food product indicated in prepared product indicia 342 is steamed. The steam cooking indicia further ties to the food product located in food container 200 by indicating a cooking operation for the food product.

Moreover, the cooking method indicia ties container 200 to sleeve 300. As indicated above in association with FIGS. 1-13, container 200 includes inner rim edge 210 and respective side walls that are structured and arranged to provide a geometric cooking indicator. As indicated in FIGS. 1-13, the geometric cooking indicator of container 200 is shaped to resemble steam vapor. As such, the steam indicator of container 200 and steam cooking indicia of sleeve 300 function in combination to drive a predetermined message related to the cooking operation and facilitate a consumer impression throughout the use of the packaging assembly. For example, the steam indicator formed by the geometric cooking indicator of container 200 signals a steam cooking operation. This steam cooking operation can be the cooking operation utilized to cook the food product in container 200. The steam cooking operation can be facilitated by several structural components of packaging assembly 100. Yet, the steaming may not be readily apparent from the components of packaging arrangement 100 that facilitate the steaming operation. The steam indicator formed by the geometric cooking indicator of container 200 provides a readily ascertainable indication that the food cooking operation includes steaming. The geometric cooking indicator of the container ties to the steam image indicia 354 and steam textual indicia 356 to reinforce the steam cooking operation throughout the use of packaging assembly 100. For example, sleeve 300 functions to drive the predetermined message of steam cooking at the point-of-sale via steam image indicia 354 and steam textual indicia 356. After container 200 is removed from sleeve 300 for use, the geometric cooking indicator of container 200 functions to drive the predetermined message.

As another example, the geometric cooking indicator of container 200 can be shaped to resemble a water droplet to indicate a boiling operation. Cooking method indicia of subfield (C1) can include boil cooking indicia such as boiling image indicia and/or boiling textual indicia. As such, the geometric cooking indicator that resembles the water droplet of container 200 and the boil cooking indicia of sleeve 300 function in combination to drive a predetermined message related to the cooking operation and facilitate a consumer impression throughout the use of the packaging assembly 100. As yet another example, the geometric cooking indicator of container 200 can be shaped to resemble a flame to indicate a grilling operation. Cooking method indicia of subfield (C1) can include grill cooking indicia such as grill image indicia and/or grill textual indicia. As such, the geometric cooking indicator that resembles the flame of container 200 and the grill cooking indicia of sleeve 300 function in combination to drive a predetermined message related to the cooking operation and facilitate a consumer impression throughout the use of packaging assembly 100. As still another example, the geometric cooking indicator of container 200 can be shaped to resemble a heat wave to indicate a conventional oven cooking operation. Cooking method indicia of subfield (C1) can include conventional oven cooking indicia such as a conventional oven image indicia and/or a conventional oven textual indicia. As such, the geometric cooking indicator that resembles the heat wave of container 200 and the conventional oven cooking indicia of sleeve 300 function in combination to drive a predetermined message related to the cooking operation and facilitate a consumer impression throughout the use of the packaging assembly

Subfield (C2) can include product type indicia. For example, as indicated in FIG. 16, the product type indicia can include textual indicia for identifying a type of food product indicated in prepared product indicia 342 and the food product located in container 200. Again, subfield (C2) is a component of brand tying indicator field (C). Accordingly, the product type indicia is ultimately tied back to brand indicia 352. The combination of subfields (C1)-(C2) of branding type indicator field (C) ties the cooking method indicia and the product type indicia to brand indicia 352 which can ultimately facilitate a nexus for brand awareness for an entire product line. For example, the nexus can drive the link between Healthy Choice® and steam cooking across a line of products.

Ingredient reinforcement indicator field (F) is arranged in coordination with the plurality of fields to reinforce a predetermined message in association with the combination of prepared product indicator field (A) and ingredient origin indicator field (B). In one aspect, ingredient reinforcement indicator field (F) overlaps ingredient origin indictor (B) so that the fields are structurally tied together. As an example associated with FIG. 16, ingredient reinforcement indicator field (F) can be populated with textual reinforcement indicia 358. As indicated, textual reinforcement indicia 358 can include the textual indicia “Fresh Taste”, which further reinforces a freshness perception related to the images associated with ingredient origin indicia 344. The combination signals a nexus between prepared product indicator 342 and the state of the ingredients indicated in prepared product indicator 342, signals a nexus between the state of the food product in container 200 and the state of the ingredients in the food product, and/or drives a predetermined message that ties a perception of freshness of the ingredients to prepared product indicator 342 and the food product in container 200.

Secondary ingredient reinforcement indicator field (E) is arranged in coordination with the plurality of fields to further reinforce a predetermined message in association with the combination of prepared product indicator field (A) and ingredient origin indicator field (B). As an example, secondary ingredient reinforcement indicator field (E) can be populated with a “cool” color to create a perception of freshness. Examples of “cool” colors can include colors, clockwise, from yellow to violet in the RYB Color Model. Yet, any color in the RYB Color Model can be utilized depending on the predetermined message. For example a “warm” color can be utilized to create a perception of spiciness. As previously indicated, secondary ingredient reinforcement indicator field (E) reinforces a predetermined message of ingredient origin indicia 344. The combination signals a nexus between prepared product indicator 342 and the state of the ingredients indicated in prepared product indicator 342, signals a nexus between the state of the food product in container 200 and the state of the ingredients in the food product, and drives a predetermined message that ties a perception of freshness of the ingredients to prepared product indicator 342 and the food product in container 200.

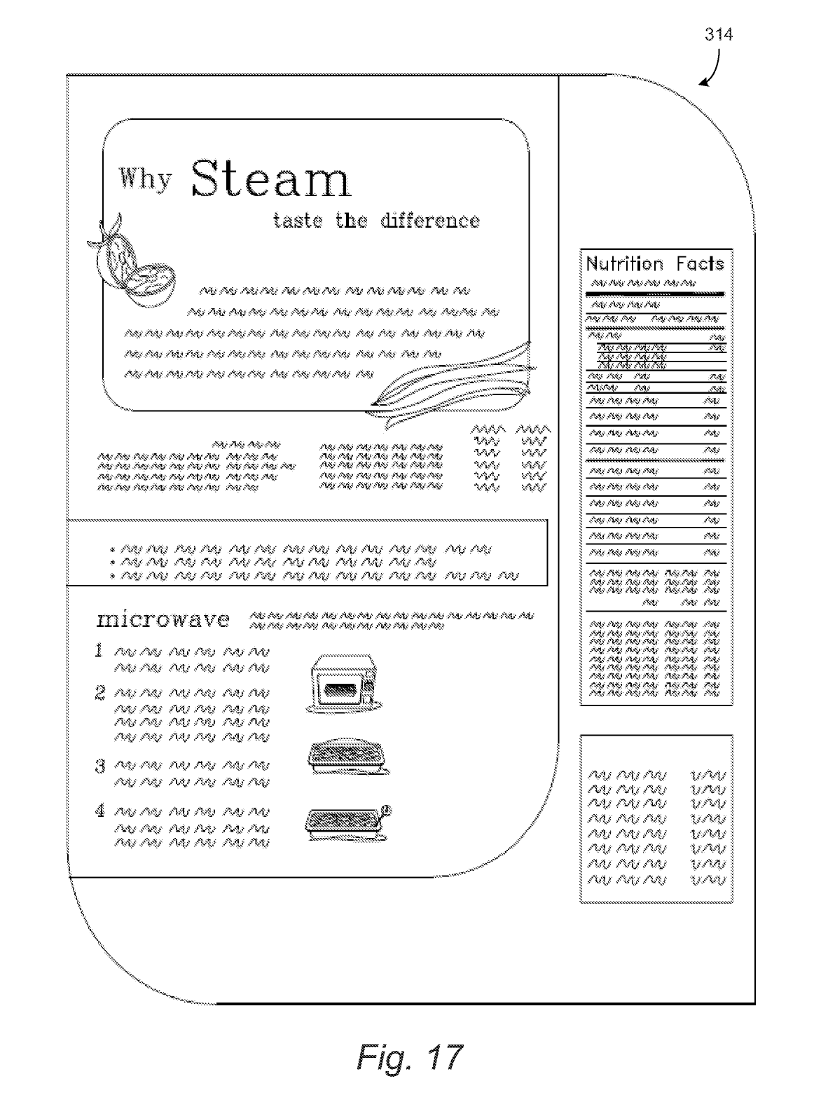

FIG. 17 is an example sleeve back 314 with example indicators populated in the indicator fields of FIG. 15. As indicated in FIG. 15, sleeve back 314 include first ingredient variant field (B1′) and second ingredient variant field (B2′). Variant fields (B1′) and (B2′) can be populated with image indicia as indicated in FIG. 17. In one aspect, the image indicia associated with (B1′) and (B2′) is an image variant associated with one or more of ingredient origin indicia 344. Even though not depicted in FIG. 17, sleeve back 314 can also include a third ingredient variant field (B3′). The nexus associated with variant fields (B1′) and (B2′) is more fully set forth above in association with ingredient origin indicia 344. FIG. 15 also includes variant field (E′). The nexus associated with variant field (E′) is more fully set forth above with respect to secondary ingredient reinforcement indicator field (E).

Sleeve back 314 includes detailed cooking method indicator field (G). Detailed cooking method indicator field (G) is arranged in coordination with the plurality of fields to provide a primary field for cooking method indicia on sleeve back 314. As an example, associated with FIG. 17, the cooking method indicia can include detailed steam dialog indicia. As indicated, detailed steam dialog indicia can include the text “Steam” along with a narrative associated with steam cooking. The cooking method indicia of sleeve back 314 creates a nexus between prepared product indicia 342 and the cooking method indicia. For example, the cooking method indicia in subfield (G) indicates steam cooking. The steam cooking indication ties to prepared product indicia 342 by indicating that the food product indicated in the prepared product indicia 342 is steamed. The steam cooking indicia further ties to the food product located in food container 200 by indicating a method of preparation of the food product. Moreover, the cooking method indicia ties container 200 to sleeve 300. As indicated above in association with FIGS. 1-13, container 200 includes inner rim edge 210 and respective side walls that are structured and arranged to provide a steam indicator. As indicated in FIGS. 1-13, the steam indicator of container 200 is shaped to resemble a steam vapor. As such, the steam indicator of container 200 and steam cooking indicia of sleeve 300 function in combination to drive a predetermined message and facilitate a consumer impression throughout the use of the packaging assembly. For example, sleeve 300 functions to drive the predetermined message at the point-of-sale. After container 200 is removed from sleeve 300 for use, the steam indicator of container 200 functions to drive the predetermined message.

Sleeve back 314 can further include first nutritional indicator field (H), product preparation method indicator field (I), secondary nutritional indicator field (J), and dietary program indicator field (K). As indicated in FIG. 17, first nutritional indicator field (H) can be populated with textual indicia that indicates food safety information. Product preparation method indicator field (I), can be populated with textual and image indicia that sets forth a method for preparing the food product located in container 200. Secondary nutritional indicator field (J) can be populated with textual nutritional fact indicia. Dietary program indicator filed (K), can be populated with information associated with a dietary program that is related to the food product located in container 200.

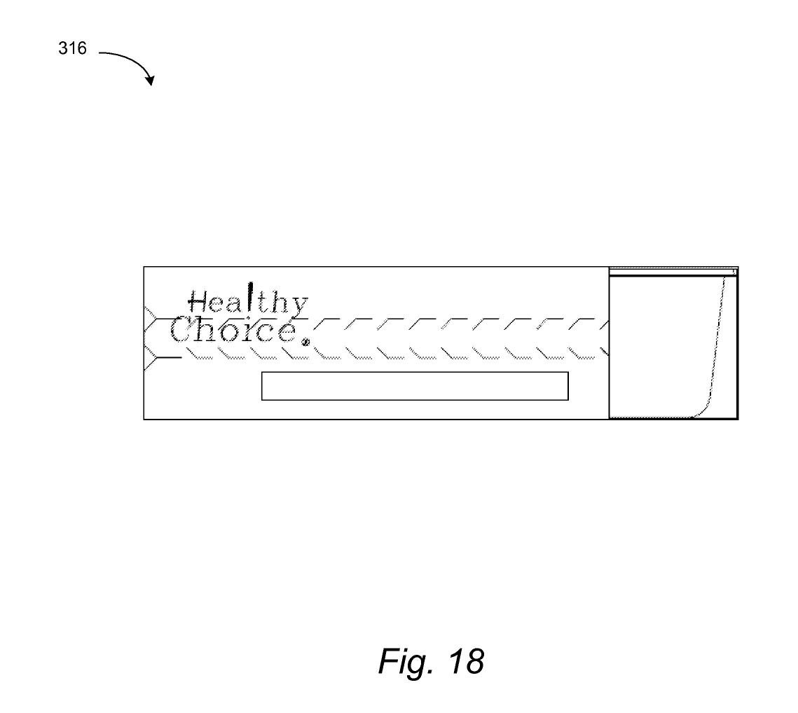

FIG. 18 is an example sleeve first side 316 with example indicators populated in the indicator fields of FIG. 15. Brand variant indicator field (D′) can include indicia that is the same as or a variant of brand indicator field (D). Example indicia are indicated in FIG. 18. The example indicia indicated in FIG. 18 can cause a similar nexus as described above in association with brand indicator field (D). Even though not depicted in FIG. 18, sleeve first side 316 can also include a variant field of secondary ingredient reinforcement indicator field (E).

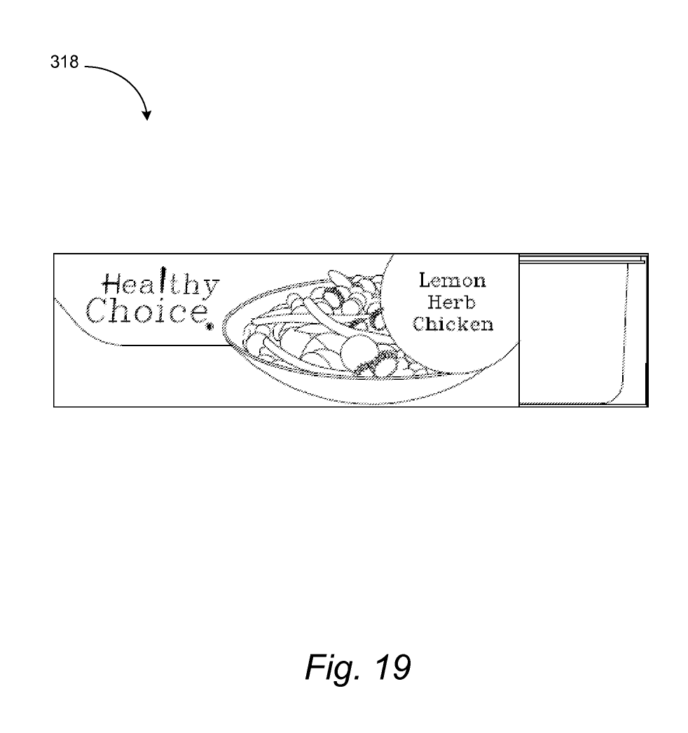

FIG. 19 is an example sleeve second side 318 with example indicators populated in the indicator fields of FIG. 15. Sleeve second side 318 also includes a brand variant indicator field (D′). FIG. 19 further includes prepared product variant indicator field (A′) and product type variant indicator field (C2′). Example indicia populated to the fields are indicated in FIG. 19. The example indicia indicated in FIG. 19 can cause a similar nexus as described above in association with brand indicator field (D), product variant indicator field (A) and product type variant indictor field (C2), respectively. Even though not depicted in FIG. 19, sleeve second side 318 can also include a variant field of secondary ingredient reinforcement indicator field (E). Sleeve second side 318, can also include a variant to ingredient reinforcement indicator field (F).

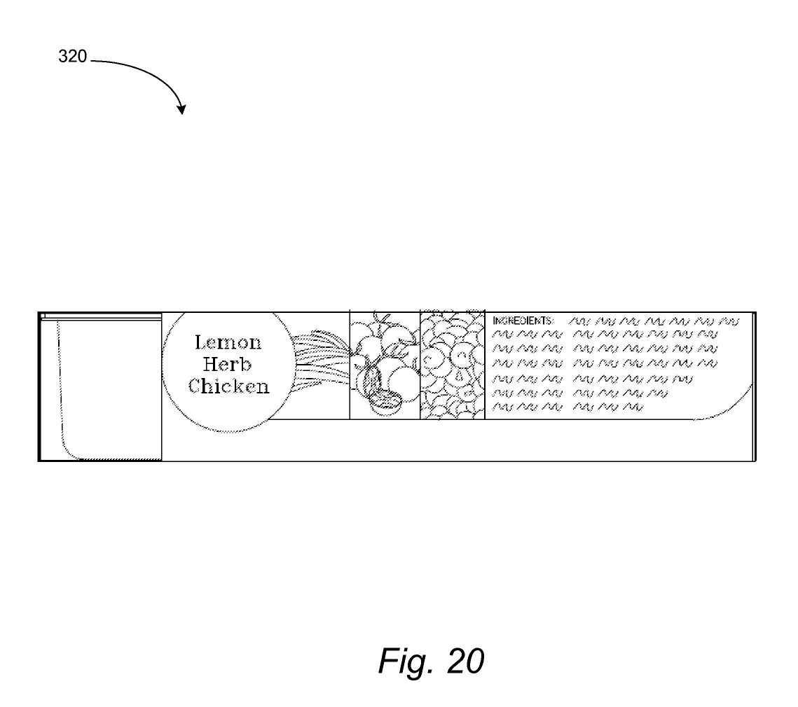

FIG. 20 is an example sleeve third side 320 with example indicators populated in the indicator fields of FIG. 15. FIG. 15 includes product type variant field (C2′) as indicated above. FIG. 15 also includes ingredient origin variant indicator field (B) having a variant first ingredient indicator (B1′), a variant second ingredient indicator field (B2′), and a variant third ingredient indicator field (B3′). The example indicia indicated in FIG. 20 can cause a similar nexus as described above in association with product type field (C2) and ingredient origin indicator field (B), respectively. Even though FIG. 20 indicates a particular order of the arrangement, the indicators can be arranged in other orders and depart from the disclosure herein. Even though not depicted in FIG. 20, sleeve third side 320 can also include a variant field of secondary ingredient reinforcement indicator field (E).

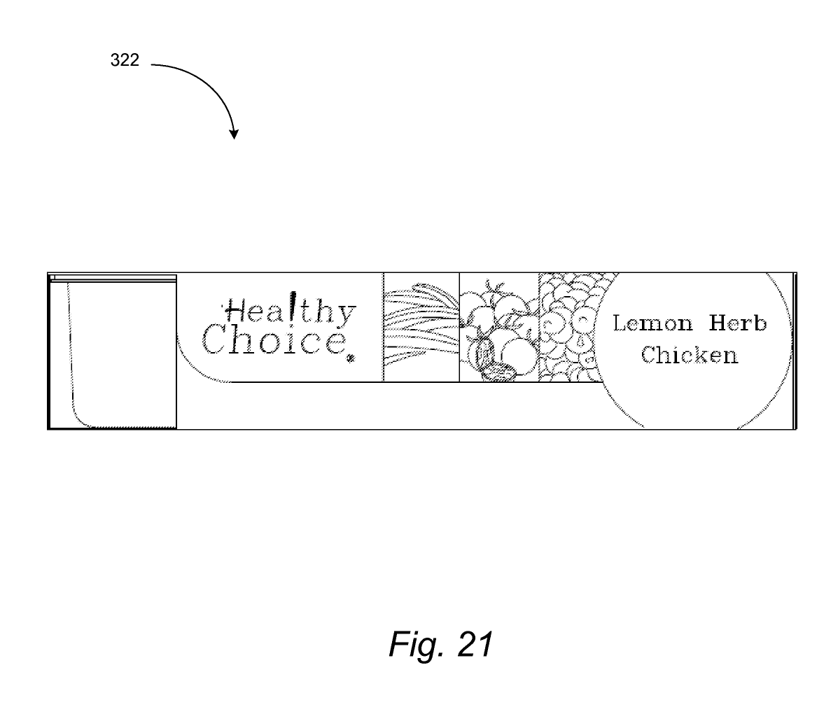

FIG. 21 is an example sleeve fourth side 322 with example indicators populated in the indicator fields of FIG. 15. Sleeve fourth side 322 also includes a brand variant indicator field (D′), product type variant field (C2′), and variant field (E′). Sleeve fourth side 322 further includes ingredient origin variant indicator field having a variant first ingredient indicator (B1′), a variant second ingredient indicator field (B2′), and a variant third ingredient indicator field (B3′). The example indicia indicated in FIG. 21 can cause a similar nexus as described above in association with brand indicator field (D), product type field (C2), secondary ingredient reinforcement indicator field (E) and ingredient origin indicator field (B), respectively. Even though not depicted in FIG. 21, sleeve fourth side 322 can also include a variant field of secondary ingredient reinforcement indicator field (E).

Packaging Nexus

As set forth above, packaging assembly 100 includes container 200 and sleeve 300 having a combination of components to drive a predetermined message to facilitate a consumer impression. An example is set forth herein to illustrate an example nexus formed between some of the components of the packaging assembly 100. The example is not meant to limit the disclosure in any manner or limit the combination of components of packaging assembly 100 to any particular combination for providing such a nexus for the predetermined message. Furthermore, even though predetermined message examples related to a perception of “freshness” and “steam” are indicated herein, the predetermined message can be associated with any type of message which the manufacturer desires to convey about a product in container 200 to facilitate a consumer impression.

As an example, a manufacturer may desire conveying a predetermined message to cause a perception that a food product is made with fresh ingredients and to indicate that the food product is steam cooked to provide a desirable healthy meal. The manufacturer may desire conveying such a message from the point-of-sale to the ultimate consumption of the food product. A packaging assembly 100 is configured as indicated above with respect to FIGS. 1-21. Packaging assembly 100 with the associated food product is shipped to a point-of-sale. Packaging assembly 100 is positioned on a shelf at the point-of-sale in an upright position as indicated in FIG. 1. The upright position is partially maintained by second closed corner 308 which provides a stable base for packaging assembly 100.

In the upright position as indicated in FIG. 1, sleeve front 312 provides a primary viewable surface to any potential customer. As indicated in FIG. 16, a predetermined message to create a perception of “freshness” is driven by the nexus indicated on sleeve front 312. Ingredient origin indicia 344 are arranged adjacent prepared product indicia 342. Ingredient origin indicia 344 includes first ingredient origin indicia 346, second ingredient origin indicia 348, and third ingredient origin indicia 350. Again, first ingredient origin indicia 346, second ingredient origin indicia 348, and third ingredient origin indicia 350 can include an array associated with ingredients indicated in prepared product indicia 342 and the food product located in container 200.

For example, container 200 can include a food product that includes green beans as an ingredient of a food product in a packaged state. Prepared product indicia 342 indicates the green beans in a cooked state and first ingredient origin indicia 344 indicates the green beans in a prepackaged state. Container 200 also includes, for example, a food product that includes tomatoes as an ingredient of a food product in a packaged state. Prepared product indicia 342 indicates the tomatoes in a cooked state and second ingredient origin indicia 348 indicates the tomatoes in a or prepackaged state. Container 200 further includes, for example, a food product that includes a starch as an ingredient of a food product in a packaged state. Prepared product indicia 342 indicates the starch in a cooked state and third ingredient origin indicia 350 indicates the starch in a prepackaged state.

The predetermined “freshness” perception message is further accentuated by textual reinforcement indicia 358 that includes the textual indicia “Fresh Taste” partially overlapping ingredient origin indicia 344, which further reinforces the images associated with ingredient origin indicia 344. Also, secondary ingredient reinforcement indicator field (E) is populated with a “cool” color. In this example, the secondary ingredient reinforcement indicator includes a blue hued color. The combination of prepared product indicia 342, ingredient origin indicia 344, textual reinforcement indicia 358 and the secondary ingredient reinforcement indicator (E) signals a nexus between prepared product indicator 342 and the state of the ingredients indicated by prepared product indicator 342, signals a nexus between the state of the food product in container 200 and the state of the ingredients in the food product, and drives the predetermined message that ties a perception of freshness of the ingredients to prepared product indicator 342 and the food product in container 200.

Again, in the upright position as indicated in FIG. 1, sleeve front 312 provides a primary viewable surface to any potential customer. As indicated in FIG. 16, a predetermined message of “steaming” is also driven by the nexus indicated on sleeve front 312. As indicated, the predetermined “steaming” message is driven by steam image indicia 354 and steam textual indicia 356. Steam image indicia 354 includes a steam vapor and steam textual indicia 356 includes the text “Steam”. The cooking method indicia creates a link to prepared product indicia 342. For example, the cooking method indicia indicates steam cooking. The steam cooking indication ties to product indicia 342 by indicating that the food product indicated in the product indicia 342 is steamed. The steam cooking indicia further ties to the food product located in food container 200 by indicating a method of preparation of the food product.

A consumer at the point-of-sale navigates packaging assembly 100. As indicated in FIG. 1, first corner opening 302 and second corner opening 304 provide a window through sleeve 300 for viewing container 200 and the food product located in container 200. Also, as indicated above, container 200 geometrically aligns with sleeve 300 at first corner opening 302 and second corner opening 304 to provide a structural nexus between container 200 and sleeve 300.

The consumer ultimately purchases the food product with packaging assembly 100. Irrespective of the positioning, the nexus indicated above is signaled to the consumer via any one of sleeve front 312, sleeve back 314, sleeve first side 316, sleeve second side 318, sleeve third side 320, or sleeve fourth side 322.

The consumer ultimately decides to consume the food product in packaging assembly 100. Sleeve 300 is reviewed for a cooking method, which is reinforced by the steaming indictors on sleeve front 312 and sleeve back 314. Sleeve 300 is then removed and discarded. Again, container 200 can be structured as indicated in FIGS. 1-13. As indicated above in association with FIGS. 1-13, container 200 includes inner rim edge 210 and respective side walls that are structured and arranged to provide a geometric cooking indicator. As indicated in FIGS. 1-13, the geometric cooking indicator of container 200 is shaped to resemble a steam vapor. As such, the geometric cooking indicator of container 200 further drives the predetermined “steaming” message and further supports a consumer impression after sleeve 300 has been removed. For example, sleeve 300 functions to drive the predetermined “steaming” message at the point-of-sale. After container 200 is removed from sleeve 300 for use, the steam indicator of container 200 functions to drive the predetermined “steaming” message.

The consumer returns to the point-of-sale for a second time and attempts to recall the food product. The consumer searches the aisles and encounters the food product. Not only does sleeve 300 facilitate a recollection of the food product, but the nexus at first corner opening 302 and second corner opening 304 created by the geometric alignment between sleeve 300 and container 200 at the first and second corner openings signals a recollection of container 200 of the product which is ultimately tied, via the nexus, to sleeve 300.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

We claim:

1. A packaging assembly, comprising:
a packaging sleeve having a sleeve front and a sleeve back, wherein the packaging sleeve includes:
a first curved corner opening, wherein the first curved corner opening is formed from a portion of a circumference of an ellipse having a first radius, and
a second curved corner opening, wherein the second curved corner opening is formed form a portion of a circumference of an ellipse having a second radius;
a container, wherein the packaging sleeve at least partially encloses the container, wherein the container includes a rim outer edge, wherein the rim outer edge includes:
a first corner curve, wherein the first corner curve is formed from a portion of a circumference of an ellipse having a third radius, and
a second corner curve, wherein the second corner curve is formed from a portion of a circumference of an ellipse having a fourth radius;
wherein the first radius is generally the same as the third radius to cause a geometrical alignment between the first curved corner opening of the packaging sleeve and the first corner curve of the container, wherein the second radius is generally the same as the fourth radius to cause a geometrical alignment between the second curved corner opening of the packaging sleeve and the second corner curve of the container.

2. The packaging assembly of claim 1, wherein the first curved corner opening of the packaging sleeve is diagonally oriented in relation to the second curved corner opening of the packaging sleeve, wherein the first corner curve of the rim outer edge is diagonally oriented in relation to the second corner curve of the rim outer edge.

3. The packaging assembly of claim 1, wherein the packaging sleeve further comprises a first closed corner and a second closed corner.

4. The packaging assembly of claim 1, wherein the length of the first radius, the length of the second radius, the length of the third radius, and the length of the fourth radius are generally the same.

5. The packaging assembly of claim 1, wherein the container rim further includes a rim inner edge, wherein the rim inner edge includes:
a first side inner rim edge,
a second side inner rim edge,
a third side inner rim edge, wherein the third side inner rim edge includes:
a third side convex curve formed from a portion of a circumference of an ellipse having a fifth radius, and
a third side concave curve formed from a portion of a circumference of an ellipse having a sixth radius,
a fourth side inner rim edge, wherein the fourth side inner rim edge includes:
a fourth side convex curve formed from a portion of a circumference of an ellipse having a seventh radius, and
a fourth side concave curve formed from a portion of a circumference of an ellipse having an eighth radius;

6. The packaging assembly of claim 5, wherein the length of the fifth radius is greater than at least one member of a group comprising: the length of the sixth radius and the length of the eighth radius.

7. The packaging assembly of claim 5, wherein the length of the seventh radius is greater than at least one member of a group comprising: the length of the sixth radius and the length of the eighth radius.

8. The packaging assembly of claim 5, wherein the length of the fifth radius is generally the same as the length of the seventh radius, wherein the length of the sixth radius is generally the same as the length of the eighth radius.

9. The packaging assembly of claim 5, wherein the rim inner edge further comprises:
a first corner curve geometrically transitioning the third side concave curve to the first side inner rim edge, wherein the first corner curve is formed from a portion of a circumference of an ellipse having a ninth radius, and
a second corner curve geometrically transitioning the fourth side concave curve to the second side inner rim edge, wherein the second corner curve is formed from a portion of a circumference of an ellipse having a tenth radius.

10. The packaging assembly of claim 9, wherein the length of the ninth radius is generally the same as the length of the tenth radius.

11. The packaging assembly of claim 9, wherein the length of the ninth radius is at least one member of a group comprising: less than the length of the fifth radius, less than the length of the sixth radius, less than the length of the seventh radius, and less than the length of the eighth radius.

12. The packaging assembly of claim 1, wherein the sleeve front includes a prepared product indicator field and an ingredient indicator field.

13. The packaging assembly of claim 12, wherein the prepared product indicator field is populated with a prepared product indicator that includes image indicia signaling a state of a cooked food product having at least one cooked ingredient.

14. The packaging assembly of claim 13, wherein the ingredient indictor field is populated with at least one ingredient indicator that includes image indicia signaling a prepackaged state of the at least one cooked ingredient signaled by the prepared product indicator field.

15. The packaging assembly of claim 14, wherein the sleeve front further includes a primary ingredient reinforcement indicator field.

16. The packaging assembly of claim 15, wherein the primary ingredient reinforcement indicator field overlaps the ingredient indicator field.

17. The packaging assembly of claim 15, wherein the primary ingredient reinforcement indicator field is populated with textual indicia signaling the preprocessed state of the image indicia populated to the ingredient indicator field.

18. The packaging assembly of claim 17, wherein the sleeve front further includes a secondary ingredient reinforcement indicator field.

19. The packaging assembly of claim 18, wherein the secondary ingredient reinforcement indicator field is populated with a cool color.

20. The packaging assembly of claim 19, wherein the cool color is a color, clockwise from yellow to violet in the RYB Color Model.

21. The packaging assembly of claim 12, wherein the prepared product indicator field is populated with a prepared product indicator that includes image indicia signaling a state of a cooked food product having a plurality of cooked ingredients.

22. The packaging assembly of claim 21, wherein the ingredient indictor field is populated with an array of ingredient indicators, wherein each ingredient indicator in the array includes image indicia signaling a prepackaged state of at least one of the cooked ingredients in the plurality of cooked ingredients.

23. The packaging assembly of claim 1, wherein the sleeve front includes a brand indicator field and a brand tying indicator field.

24. The packaging assembly of claim 23, wherein the brand indicator field is populated with textual indicia that signals a brand of a food product.

25. The packaging assembly of claim 24, wherein the brand tying indicator field is populated with image indicia having a shape corresponding to at least one portion of the image indicia populated to the brand indicator field.

26. The packaging assembly of claim 23, wherein the brand tying indicator field is populated with at least one member of a group comprising: image indicia signaling a cooking method for a food product, textual indicia signaling a cooking method for a food product, and textual indicia signaling a product type for a food product.

27. The packaging assembly of claim 24, wherein the brand tying indicator field includes a first sub-field and a second sub-field, wherein the first sub-field and the second sub-field are graphically separate, wherein the first sub-field and the second sub-field are populated with image indicia having a shape that collaboratively corresponds to at least one portion of the image indicia populated to the brand indicator field.

28. A product assembly, comprising:
a food product located in a container, wherein the food product includes a plurality of ingredients;
a packaging sleeve enclosing at least a portion of the container;
the packaging sleeve having a sleeve front and a sleeve back, wherein the packaging sleeve includes:
a curved corner opening, wherein the curved corner opening is formed from a portion of a circumference of an ellipse having a first radius;
the container includes a rim outer edge, wherein the rim outer edge includes:
a corner curve, wherein the corner curve is formed from a portion of a circumference of an ellipse having a second radius;
wherein the first radius is generally the same as the second radius to cause a geometrical alignment between the curved corner opening of the packaging sleeve and the corner curve of the container.

29. The product assembly of claim 28, wherein the packaging sleeve further comprises a first closed corner and a second closed corner.

30. The product assembly of claim 28, wherein the length of the first radius and the length of the second radius are generally the same.

31. The product assembly of claim 28, wherein the sleeve front includes a prepared product indicator field and an ingredient indicator field.

32. The product assembly of claim 31, wherein the prepared product indicator field is populated with a prepared product indicator that includes image indicia signaling a cooked stated of the food product located in the container.

33. The product assembly of claim 32, wherein the ingredient indictor field is populated with an array of ingredient indicators that include image indicia signaling a prepackaged state of the plurality of ingredients of the frozen meal located in the container.

34. The product assembly of claim 33, wherein the sleeve front further includes a primary ingredient reinforcement indicator field.

35. The product assembly of claim 34, wherein the primary ingredient reinforcement indicator field overlaps the ingredient indicator field.

36. The product assembly of claim 34, wherein the primary ingredient reinforcement indicator field is populated with textual indicia that signals at least one member of a group comprising: the preprocessed state of the plurality of ingredients of the food product located in the container, the preprocessed state of the array of ingredient indicators populated to the ingredient indicator field, and the prepackaged state of the prepared product indicator populated to the prepared product indicator field.

37. The product assembly of claim 36, wherein the sleeve front further includes a secondary ingredient reinforcement indicator field.

38. The product assembly of claim 37, wherein the secondary ingredient reinforcement indicator field is populated with a cool color, wherein the cool color is a color, clockwise from yellow to violet in the RYB Color Model.

39. A packaging assembly, comprising:
a packaging sleeve, wherein the packaging sleeve includes:
a sleeve front, wherein the sleeve front includes:
a first front curve formed from a portion of a circumference of an ellipse having a first radius, and
a second front curve formed from a portion of a circumference of an ellipse having a second radius,
a sleeve back, wherein the sleeve back includes:
a first back curve formed from a portion of a circumference of an ellipse having a third radius, and
a second back curve formed from a portion of a circumference of an ellipse having a fourth radius;
wherein the first front curve and the first back curve form a first corner opening, and
wherein the second front curve and the second back curve form a second corner opening;
a container, wherein the packaging sleeve encloses at least a portion of the container, wherein the container includes a rim outer edge, wherein the rim outer edge includes:
a first corner curve, wherein the first corner curve is formed from a portion of a circumference of an ellipse having a fifth radius, and
a second corner curve, wherein the second corner curve is formed from a portion of a circumference of an ellipse having a sixth radius;
wherein the fifth radius is generally the same as the first radius and the third radius to cause a geometrical alignment between the first curved corner opening of the packaging sleeve and the first corner curve of the container, wherein the sixth radius is generally the same as the second radius and the fourth radius to cause a geometrical alignment between the second curved corner opening of the packaging sleeve and the second corner curve of the container.

40. The packaging assembly of claim 39, wherein the first curved corner opening of the packaging sleeve is diagonally oriented in relation to the second curved corner opening of the packaging sleeve, wherein the first corner curve of the rim outer edge is diagonally oriented in relation to the second corner curve of the rim outer edge.

41. The packaging assembly of claim 39, wherein the packaging sleeve further comprises a first closed corner and a second closed corner.

42. The packaging assembly of claim 39, wherein the container rim further includes a rim inner edge, wherein the rim inner edge includes:
a first side inner rim edge,
a second side inner rim edge,
a third side inner rim edge, wherein the third side inner rim edge includes:
a third side convex curve formed from a portion of a circumference of an ellipse having a seventh radius, and
a third side concave curve formed from a portion of a circumference of an ellipse having an eighth radius,
a fourth side inner rim edge, wherein the fourth side inner rim edge includes:
a fourth side convex curve formed from a portion of a circumference of an ellipse having a ninth radius, and
a fourth side concave curve formed from a portion of a circumference of an ellipse having a tenth radius;

43. The packaging assembly of claim 42, wherein the length of the seventh radius is greater than at least one member of a group comprising: the length of the eighth radius and the length of the tenth radius.

44. The packaging assembly of claim 42, wherein the length of the ninth radius is greater than at least one member of a group comprising: the length of the eighth radius and length of the tenth radius.

45. The packaging assembly of claim 42, wherein the length of the seventh radius is generally the same as the length of the ninth radius, wherein the length of the eighth radius is generally the same as the length of the tenth radius.
